(19) United States
(12) Patent Application Publication
(10) Pub. No.: US 2023/0255853 A1
(43) Pub. Date: Aug. 17, 2023

(54) MEDICATION SUPPORT DEVICE

(71) Applicants: Hiroshi Fujie, Kanagawa (JP); Hirotaka Hayashi, Kanagawa (JP); Manabu Yamanaka, Kanagawa (JP); Youhei Niitsuma, Kanagawa (JP); Yu Yamaya, Kanagawa (JP)

(72) Inventors: Hiroshi Fujie, Kanagawa (JP); Hirotaka Hayashi, Kanagawa (JP); Manabu Yamanaka, Kanagawa (JP); Youhei Niitsuma, Kanagawa (JP); Yu Yamaya, Kanagawa (JP)

(73) Assignee: Ricoh Company, Ltd., Tokyo (JP)

(21) Appl. No.: 18/151,127

(22) Filed: Jan. 6, 2023

(30) Foreign Application Priority Data
Feb. 15, 2022 (JP) 2022-021639

(51) Int. Cl. A61J 7/00; G16H 20/13

(52) U.S. Cl. CPC A61J 7/0084; G16H 20/13

(57) ABSTRACT

A medication support device includes a container that stores multiple medicine packs stacked on top of each other in layers, a pickup device to pick up a specific one of the multiple medicine packs from the container, a conveyor to convey the specific one of the multiple medicine packs picked up by the pickup device, a medicine dispenser to arrange the specific one of the multiple medicine packs conveyed by the conveyor, and a pack passing-through portion disposed on a bottom of the container. In the medication support device, the specific one of the multiple medicine packs picked up by the pickup device passes through the pack passing-through portion, and the pack passing-through portion has multiple support portions that support the multiple medicine packs. In the medication support device, the multiple support portions are fixation members fixed to the pack passing-through portion, and the multiple support portions vary in shape.

### CROSS-REFERENCE TO RELATED APPLICATION

This patent application is based on and claims priority pursuant to 35 U.S.C. § 119(a) to Japanese Patent Application No. 2022-021639, filed on Feb. 15, 2022, in the Japan Patent Office, the entire disclosure of which is hereby incorporated by reference herein.

### BACKGROUND

### Technical Field

Embodiments of the present disclosure relate to a medication support device.

### Background Art

For the purpose of reducing the time or labor of medication, medication support devices are known in the art. More specifically, some technologies have been proposed to pass the picked-up pack to a medicine dispensing tray after picking up a pack from a container in which a plurality of medicine packages packing a several kinds of medicines are accommodated using an adsorption system. In such proposed technologies, the medicine dispensing tray is then ejected to the exit of the housing of the medication support device, and the medicine is passed to a person who takes the medicine or a medication assistant.

### SUMMARY

Embodiments of the present disclosure described herein provide a medication support device includes a container that stores a plurality of medicine packs stacked on top of each other in layers, a pickup device to pick up a specific one of the multiple medicine packs from the container, a conveyor to convey the specific one of the multiple medicine packs picked up by the pickup device, a medicine dispenser to arrange the specific one of the multiple medicine packs conveyed by the conveyor, and a pack passing-through portion disposed on a bottom of the container. In the medication support device, the specific one of the multiple medicine packs picked up by the pickup device passes through the pack passing-through portion, and the pack passing-through portion has a plurality of support portions that support the multiple medicine packs. In the medication support device, the multiple support portions are fixation members fixed to the pack passing-through portion, and the multiple support portions vary in shape.

### BRIEF DESCRIPTION OF THE DRAWINGS

A more complete appreciation of embodiments and the many attendant advantages thereof will be readily obtained as the same becomes better understood by reference to the following detailed description when considered in connection with the accompanying drawings.

FIG. 1A is a front view of a medication support device in its entirety, according to an embodiment of the present disclosure.

FIG. 1B is a side view the medication support device of FIG. 1A in its entirety, according to an embodiment of the present disclosure.

FIG. 2A is a typical plan view of a single medicine package according to an embodiment of the present disclosure.

FIG. 2B a side view of the single medicine package of FIG. 2A viewed in a direction indicated by an arrow A, according to an embodiment of the present disclosure.

FIG. 2C is a perspective view of bound packages according to an embodiment of the present disclosure.

FIG. 2D is a view of a typical form of continuous packs according to an embodiment of the present disclosure.

FIG. 3 is an external perspective view of a medicine dispensing tray, illustrating one configuration of the medicine dispensing tray, according to an embodiment of the present disclosure.

FIG. 4A is a vertical sectional view of a cartridge according to an embodiment of the present disclosure.

FIG. 4B is a bottom view of a cartridge according to an embodiment of the present disclosure.

FIG. 5 is a vertical sectional view of the cartridge of FIG. 4A, which is magnified for view and exaggerated for purposes of illustration, according to an embodiment of the present disclosure.

FIG. 6 is a schematic plan sectional view of the attaching and detaching mechanisms for a container, which are arranged on a drawer, according to an embodiment of the present disclosure.

FIG. 7 is a plan view of a drawer illustrating the mechanisms for recognizing a container, which are arranged on the drawer, according to an embodiment of the present disclosure.

FIG. 8A is a front view of a pickup unit, illustrating a configuration or structure of the pickup unit, according to an embodiment of the present disclosure.

FIG. 8B is a plan view of the pickup unit illustrated in FIG. 8A, according to an embodiment of the present disclosure.

FIG. 9A, FIG. 9B, FIG. 9C, FIG. 9D, FIG. 9E, and FIG. 9F are front views a pickup unit illustrating the progression of the operation of the pickup unit, according to an embodiment of the present disclosure.

FIG. 10A and FIG. 10B are front views of the pickup unit illustrating the progression of the operation of the pickup unit subsequent to the progression illustrated in FIG. 9A, FIG. 9B, FIG. 9C, FIG. 9D, FIG. 9E, and FIG. 9F, according to an embodiment of the present disclosure.

FIG. 11A is a front view of a conveyance unit illustrating a schematic configuration of the conveyance unit, according to an embodiment of the present disclosure.

FIG. 11B is a side view of the conveyance unit illustrated in FIG. 11A, according to an embodiment of the present disclosure.

FIG. 12 is a control block diagram illustrating a schematic control structure for a medication support device according to an embodiment of the present disclosure.

FIG. 13A-1, FIG. 13A-2, FIG. 13A-3, FIG. 13A-4, FIG. 13A-5, FIG. 13A-6, FIG. 13B, and FIG. 13C are schematic diagrams each illustrating an overall operation flow of the medication support device illustrated in FIG. 1, according to an embodiment of the present disclosure.

FIG. 14 is a flowchart of the operation flow of a pickup unit according to an embodiment of the present disclosure.

FIG. 15A is a vertical sectional view of a cartridge according to a modification of the above embodiments of the present disclosure.

FIG. 15B is a bottom view of the cartridge of FIG. 15A according to a modification of the above embodiments of the present disclosure.

FIG. 16A is a vertical sectional view of the cartridge according to a modification of the above embodiments of the present disclosure different from the modifications illustrated in FIG. 15A and FIG. 15B.

FIG. 16B is a bottom view of the cartridge of FIG. 16A, according to an embodiment of the present disclosure.

The accompanying drawings are intended to depict embodiments of the present disclosure and should not be interpreted to limit the scope thereof. The accompanying drawings are not to be considered as drawn to scale unless explicitly noted.

### DETAILED DESCRIPTION

The terminology used herein is for the purpose of describing particular embodiments only and is not intended to be limiting of the present disclosure. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the present disclosure is not intended to be limited to the specific terminology so selected and it is to be understood that each specific element includes all technical equivalents that have the same structure, operate in a similar manner, and achieve a similar result.

Embodiments of the present disclosure and examples of the present disclosure are described below in detail with reference to the accompanying drawings. In the drawings and the description of the embodiments and examples of the present disclosure, like reference signs denote like elements such as members or components of similar shapes or similar functionality, and overlapping description may be omitted where appropriate unless there is concern about the possibility of confusion.

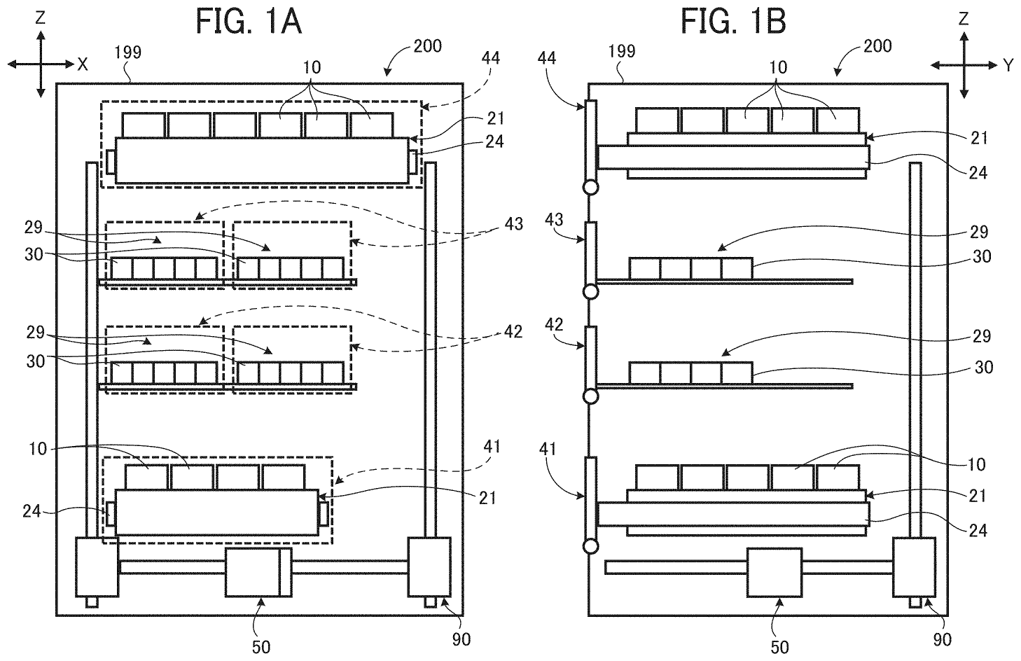

FIG. 1A is a schematic front view of a medication support device 200 in its entirety according to an embodiment of the present disclosure.

FIG. 1B is a schematic side view of the medication support device 200 of FIG. 1A, according to the present embodiment.

As illustrated in FIG. 1A and FIG. 1B, a medication support device 200 according to an embodiment of the present disclosure includes a plurality of cartridges 10 each of which is also referred to as a container, a plurality of medicine dispensing trays 30, a pickup unit 50, a conveyance unit 90, a first gate 41, a second gate 42, a third gate 43, and a fourth gate 44.

In FIG. 1A and FIG. 1B, the horizontal direction or the right and left directions of the medication support device 200 is defined as an X-direction, where the horizontal direction or the right and left directions of the medication support device 200 is also referred to as the width direction of the medication support device 200. In FIG. 1A and FIG. 1B, the front-rear direction or the depth direction of the medication support device 200 is defined as a Y-direction, and the up-and-down directions or the orthogonal direction of the medication support device 200 is defined as a Z-direction, where the up-and-down directions or the orthogonal direction of the medication support device 200 is also referred to as the vertical direction.

Each one of the multiple cartridges 10 serves as a container in which a plurality of medicine packages 2 packing a several kinds of medicines 3 as will be described later are stored upon being stacked on top of each other in layers. The medicine package 2 according to the present embodiment may be referred to simply as a pack or a medicine pack in the following description. The multiple cartridges 10 are arranged at the uppermost portion and a lower portion of a main structural frame 199 of the medication support device 200. The expression “stored upon being stacked on top of each other in layers” in the present disclosure indicates keeping the packs horizontally in such a manner that the front face is viewable.

Each one of the multiple medicine dispensing trays 30 serves as a medicine dispenser in which the prescribed packs conveyed by the conveyance unit 90 are arranged. As illustrated in FIG. 1A, four medicine dispensing trays 30 are positioned between an uppermost group of multiple cartridges 10 and a lower group of multiple cartridges 10. Hereinafter, the position of each one of the multiple medicine dispensing trays 30 where the packs are passed to the medicine dispensing tray 30 for automatic medicine dispensing is referred to as a medicine dispending position 29.

The pickup unit 50 according to the present embodiment serves as a pickup device that picks up a specific pack from one of the multiple cartridges 10. The conveyance unit 90 according to the present embodiment serves as a conveyor that conveys the pack picked up from one of the multiple cartridges 10 by the pickup unit 50.

Each one of the first gate 41 and the fourth gate 44 according to the present embodiment serve as an entrance and exit gate for the container, which allows one of the multiple cartridges 10 to enter and exit the main structural frame 199. The multiple cartridges 10 are inserted and set in the main structural frame 199 through each one of the first gate 41 and the fourth entrance 44. The open and close door of one of the first gate 41 and the fourth gate 44 are opened, and the drawer 21 in which the multiple cartridges 10 are aligned is drawn out to the front side. By so doing, the multiple cartridges 10 is inserted or withdrawn.

Each one of the second gate 42 and the third gate 43 according to the present embodiment serves as entrance and exit gate for the medicine dispenser, which allows one of the multiple medicine dispensing trays 30 to enter and exit the main structural frame 199. The second gate 42 and the third gate 43 are arranged such that the packs can be picked up immediately after the packs are set or inserted into the medicine dispensing tray 30.

In the medication support device 200 according to the present embodiment, four medicine dispensing trays 30 are arranged as illustrated in FIG. 1A. For example, a plurality of medicine dispensing trays 30 are arranged for each time of medication such as a time in the morning, a time in the daytime, a time in the evening, and a time before going to bed.

The second gate 42 and the third gate 43 for the medicine dispensing tray are also arranged for each one of the medicine dispensing trays 30. Due to such a configuration, another different one of the medicine dispensing trays 30 can be picked up even when the medicine dispersing operation is performed on a particular one of the multiple medicine dispensing trays 30.

The drawers 21 of the multiple cartridges 10 as illustrated in FIG. 1A and FIG. 1B are arranged in two stages on the upper and lower sides with the four medicine dispensing trays 30 interposed therebetween. However, no limitation is intended thereby, and all the drawers 21 may collectively be arranged on an upper side or may collectively be arranged on a lower side. Depending on the number of persons in the day-care center or nursing home or day-care center, the multiple cartridges 10 may be arranged in three stages. Such an alternative embodiment may be adopted without affecting the effects to be achieved by the embodiments of the present disclosure.

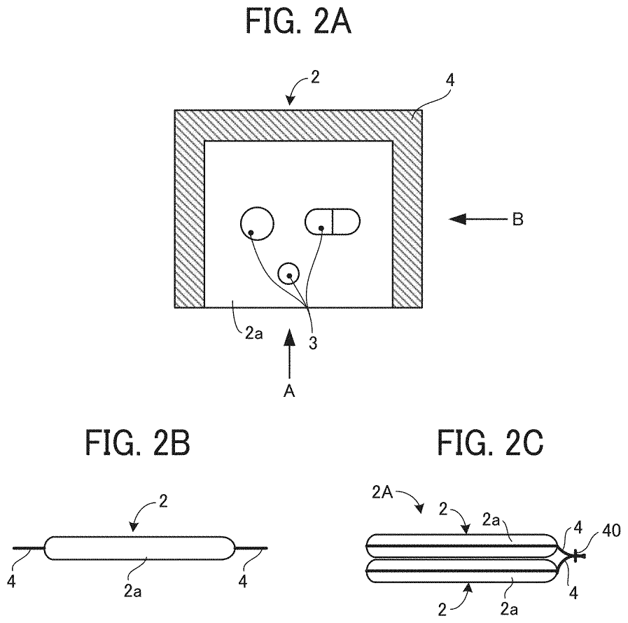

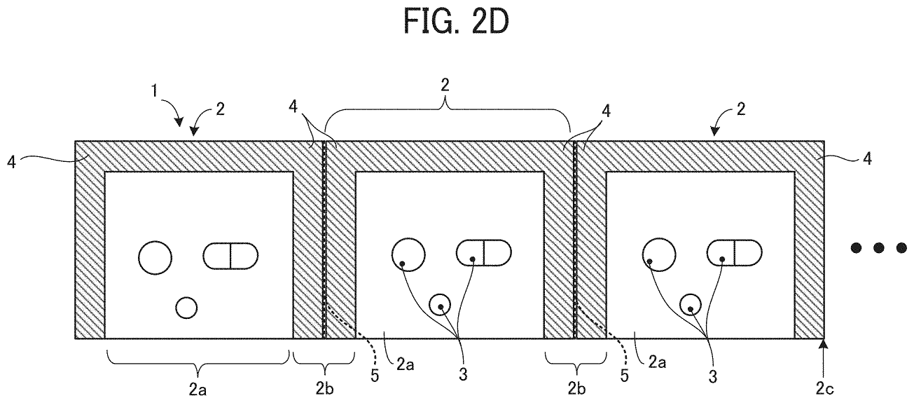

An outline of the medicine package 2 according to the present embodiment is described below with reference to FIG. 2A, FIG. 2B, FIG. 2C, and FIG. 2D.

FIG. 2A is a typical plan view of a single medicine package 2 according to the present embodiment.

FIG. 2B a side view of the single medicine package 2 of FIG. 2A viewed in a direction indicated by an arrow A, according to the present embodiment.

FIG. 2C is a perspective view of bound packages 2A according to the present embodiment.

FIG. 2D is a view of a typical form of continuous packs according to the present embodiment.

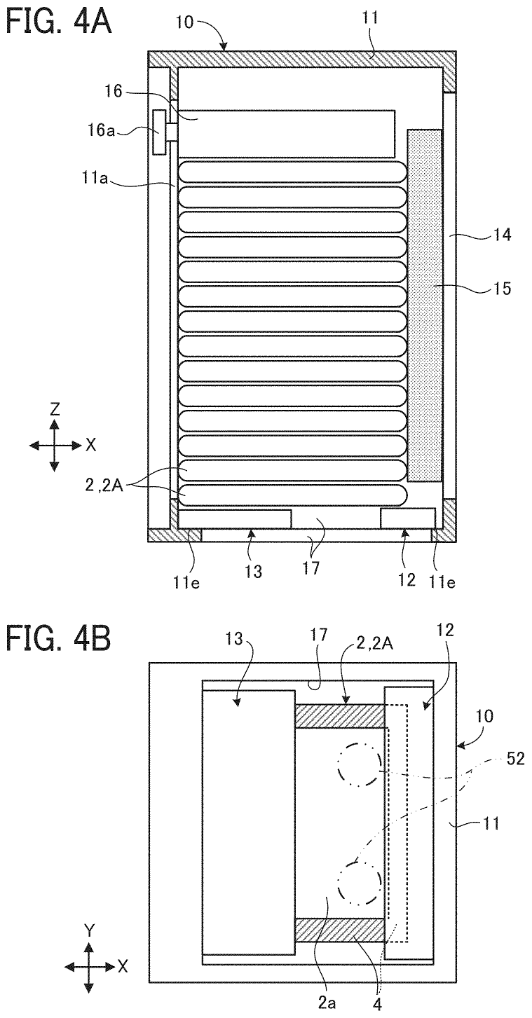

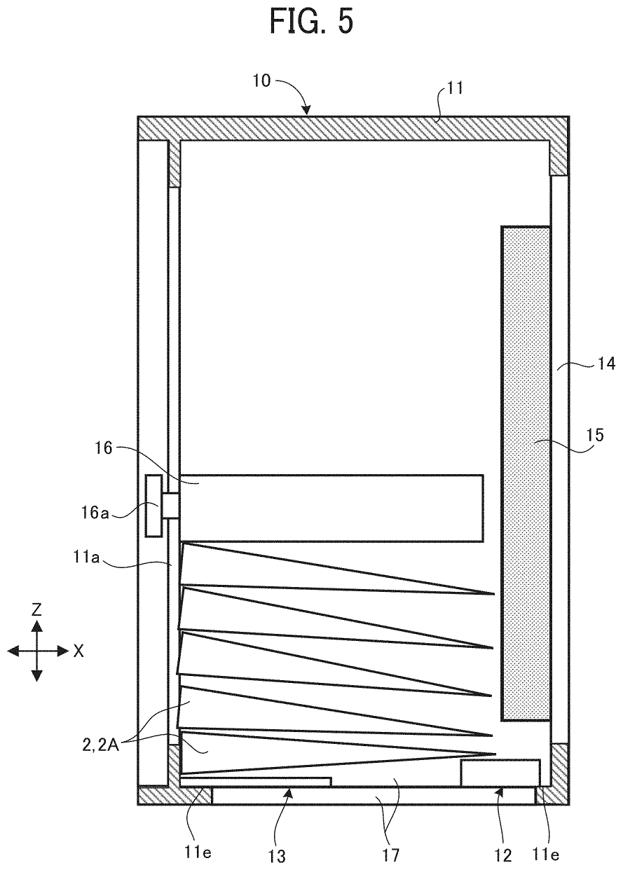

The medicine pack according to the present embodiment includes the single medicine package 2 and bound packages 2A in which a plurality of medicine packages 2 are stacked on top of each other in layers and bound by a stapler or the like. In FIG. 2C, two medicine packages 2 are bound as the bound packages 2A. The bound packages 2A according to the present embodiment may be referred to simply as bound packages 2A in the following description. Except for FIG. 2A, FIG. 2B, FIG. 2C, FIG. 2D, FIG. 4A, FIG. 4B, and FIG. 5, a single medicine package 2 is illustrated. However, as a matter of course, the bound packages 2A is also included even when only a single medicine package 2 is illustrated.

As illustrated in FIG. 2A, one medicine package 2 is formed of, for example, a resin film, and a plurality of medicines 3 such as capsules or tablets are packed in small quantities. The medicine package 2 includes a bag 2a that covers the medicines 3 and a pressure-bonded portion 4 in which three sides of the bag 2a are pressure-bonded or welded. The pressure-bonded portion 4 is hatched in FIG. 2A and FIG. 2D. Typically, the side of the medicine package 2 on the bag 2a side is folded into two, and the medicines 3 are packed therebetween. The pressure-bonded portion 4 forms a leakage preventing portion that prevents the medicines 3 from slipping out from the bag 2a. Typically, the medicines 3 in one medicine package 2 is a one-time dosage for a patient who takes medicines.

The medicine package 2 is prepared by a drug-dispensing machine installed in a pharmacy or the like. The dispensing sheets that are used for dispending the medicines are rolled long-length sheets, and are perforated between packs. The dispensing sheets are rolled in an overlapping manner, and the medicines 3 to be taken are packed in the space between the sheets. The medicines 3 for a required number of doses are divided in small quantities, and packed into the multiple medicine packages 2 that form a continuous sheet. The three sides other than the folded portion of each one of the multiple medicine packages 2 around the medicines 3 for a single dose are sealed in turn by the pressure-bonded portion 4. The multiple medicine packages 2 that form such a continuous sheet may be referred to as continuous packs 1 in the following description. In the continuous packs 1 that are described with reference to FIG. 2D, the multiple medicine packages 2 are coupled to each other and laid like a belt. In the present embodiment described with reference to FIG. 2D, the medicines 3 for three doses are illustrated. The continuous packs 1 are typical form of drug or medicine that is given or sold at a pharmacy or the like to, for example, a user such as a person who actually takes the medicine 3 in the medicine package 2 and a personal care attendant or assistant who helps or assists the user to take medication, or a staff or the like including a pharmacist, a nurse practitioner, a care manager, or a medication assistant of, for example, various kinds of day-care center, nursing home, or medical institution. For the sake of explanatory convenience, each one of the packs is filled with the medicines 3 of the same form in the following description of the present disclosure. In FIG. 2D, by way of example, each one of the packs is filled with the same capsule and the same tablets. However, no limitation is indicated thereby. As a matter of course, each one of the packs may be filled with different type of medicines depending on, for example, the usage or purposes of the medication.

In the present embodiment described with reference to FIG. 2A, FIG. 2B, FIG. 2C, and FIG. 2D, the medicine package 2 has a rectangular shape in a plan view, and a packaging method in which three sides are crimped and sealed as in the present embodiment is called three-sided packaging in the related art. Most of the drug-dispensing machines available on the market make packs using the three-sided packaging method.

The pressure-bonded portion 4 has a band-like width of about 10 to 15 millimeters (mm), and has higher rigidity than the portion of the bag 2a that is a transparent or semitransparent film where the medicines 3 can be visually recognized therethrough. A boundary portion 2b that is implemented by perforation 5 is formed in the center of the pressure-bonded portion 4 between a pair of the multiple medicine packages 2 making up the continuous packs 1. The multiple medicine packages 2 are adjacent to each other in an upstream-to-downstream direction. For example, a user who is not handicapped in the hand can obtain one medicine package 2 by tearing off at the perforation 5 by hand or by cutting near the perforation 5 with scissors or a dedicated cutter.

As illustrated in FIG. 2C, bound packages 2A may be used. The bound packages 2A are formed by fastening a plurality of medicine packages 2 at the center of the three sides of the pressure-bonded portions 4 by stapling or the like. In FIG. 2C, two medicine packages 2 are fastened together.

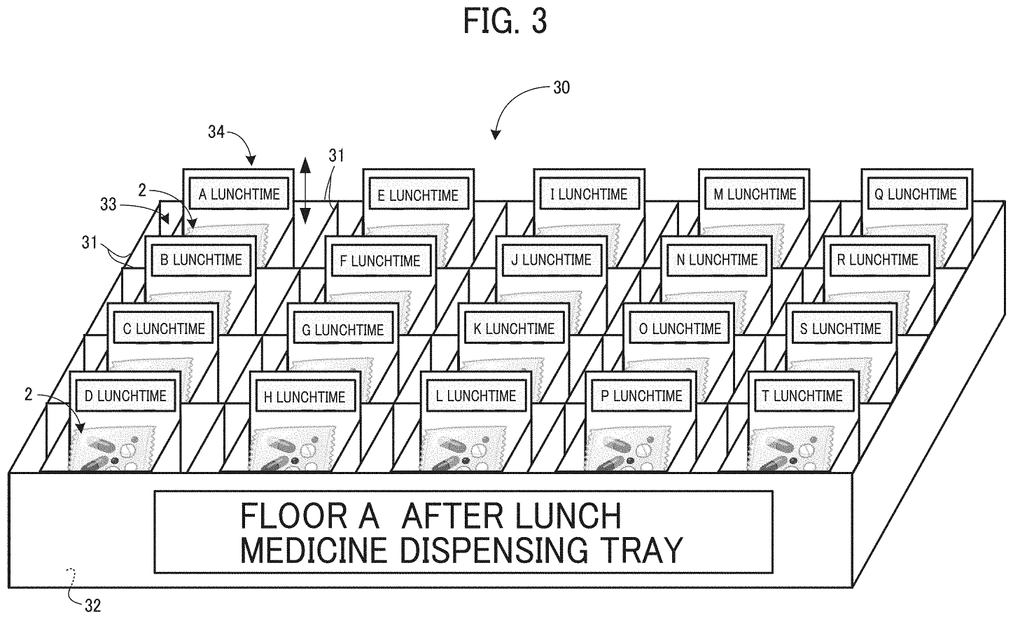

FIG. 3 is an external perspective view of the medicine dispensing tray 30, illustrating one configuration of the medicine dispensing tray 30, according to the present embodiment.

As illustrated in FIG. 3, each one of the multiple medicine dispensing trays 30 has a plurality of partition walls 31 that serve as a plurality of dividers used to arrange prescribed packs, and is partitioned into twenty partitions 33 by four upright partition walls 31. The twenty partitions 33 that are formed in each one of the multiple medicine dispensing trays 30 can be expressed as the components of a matrix including five columns in the X-direction or the line feed direction and four rows in the Y-direction or the character feed direction. As a result, each one of the twenty partitions 33 of the multiple medicine dispensing trays 30 can be uniquely positioned based on the components and addresses of the matrix of five columns and four rows. Furthermore, each one of the multiple medicine dispensing trays 30 has a bottom wall 32 on which the multiple medicine packages 2 are placed. As described above, each one of the multiple medicine dispensing trays 30 are arranged such that a specific one of the multiple packs will be placed in a specific one of the multiple partitions 33 with reliability by the multiple partition walls 31, which are four partition walls in the present embodiment, and the bottom wall 32 in common, without being mixed up with packs in different one of the multiple partitions 33 or dropping off from the bottom wall 32. FIG. 3 illustrates one of the multiple medicine dispensing trays 30 where a subdivision box 34 attachable to and detachable from each one of the multiple partitions 33 is used. For example, the multiple subdivision boxes 34 according to the present embodiment hold the multiple medicine packages 2 filled with the medicines 3 to be taken by twenty residents A to T after lunch in day-care center or nursing home. In other words, the multiple medicine dispensing trays 30 as illustrated in FIG. 3 are used to place a specific pack in a predetermined or specific one of the multiple partitions 33 partitioned by a plurality of dividers through the multiple subdivision boxes 34. The operation of placing the multiple medicine packages 2 in one of the multiple medicine dispensing trays 30 is briefly described later with a case in which the multiple subdivision boxes 34 are not used.

In each one of the multiple partitions 33 of the multiple medicine dispensing trays 30, a setting position or inserting position is determined for each patient who takes medicines depending on a type of medicines in the pack to be taken. In other words, the multiple partitions 33 in one of the multiple medicine dispensing trays 30 may be allocated to a plurality of patients who take medicines at the same time. In a case where a specific patient who takes medicines does not take a medicine at a specific timing, arrangement can be made such that a pack is not given to such a specific patient at the specific timing.

However, no limitation is intended by the multiple medicine dispensing trays 30 described above. According to an alternative embodiment of the present disclosure, each one of the multiple partitions 33 of one of the multiple medicine dispensing trays 30 may be allocated for each time of medication of each patient who takes medicines. More specifically, in such an alternative embodiment of the present disclosure, the multiple partitions 33 may be allocated to each of the patients who take medicines in view of the time of medication of each one of the multiple medicine packages 2 to be taken in the morning, in the daytime, in the evening, or before going to bed. In the multiple medicine dispensing trays 30 according to such an alternative embodiment of the present disclosure, the multiple medicine dispensing trays 30 may be managed on a room by room basis in day-care center or nursing home where a plurality of patients who take medicines reside, and the multiple medicine packages 2 for that day or a couple of days may be dispersed in advance to the multiple medicine dispensing trays 30.

According to the above alternative embodiment, allocating the partitions 33 based on each time of medication such as a time in the morning, a time in the daytime, a time in the evening, and a time before going to bed can prevent the time of medication from being mistaken by patients who take medicines. The configuration or structure of the multiple medicine dispensing trays 30 is not limited to the embodiments of the present disclosure described above, and various kinds of combination are theoretically possible between patients who take medicines and the timing at which the medicines are to be taken. However, such combinations may go beyond the scope of the present disclosure, the description of such combinations is omitted.

FIG. 4A is a vertical sectional view of the cartridge 10 according to the present embodiment.

FIG. 4B is a bottom view of the cartridge 10 of FIG. 4A, according to the present embodiment.

In the vertical sectional view of the cartridge 10 of FIG. 4A, the medicine package 2 is schematically illustrated in an enlarged and exaggerated manner.

For example, each one of the multiple cartridges 10 according to the present embodiment includes a casing 11, a lid 14, a pack pickup slot 17, a movable board 16, a pack posture keeper 15, and a right support portion 12 and a left support portion 13 each of which serves as a support portion.

The casing 11 according to the present embodiment stores, for example, a plurality of medicine packages 2 or the bound packages 2A. In the following description, a single medicine package 2 will represent such a plurality of medicine packages 2 or bound packages 2A. For example, the casing 11 is integrally or separately formed using resin.

The lid 14 according to the present embodiment enables the multiple medicine packages 2 to be loaded or unloaded.

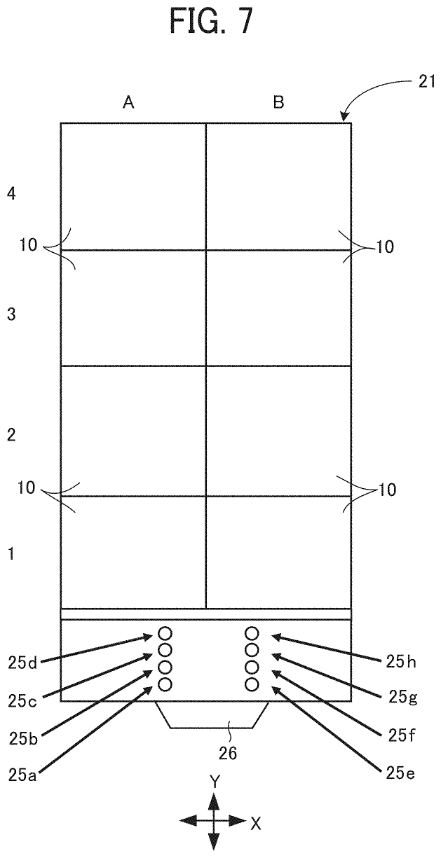

The pack pickup slot 17 according to the present embodiment is formed in a lower portion or a bottom portion of the casing 11, and is used to pick up the medicine package 2 in one of the multiple cartridges 10. Moreover, the pack pickup slot 17 according to the present embodiment allows the medicine package 2, which is picked up from one of the multiple cartridges 10 by the pickup unit 50 (see, for example, FIG. 1A, FIG. 1B, and FIG. 7), to pass therethrough.

The movable board 16 according to the present embodiment serves as a pressing member and prevents the medicine package 2 from falling out, and makes the lowermost one of the multiple medicine packages 2 move close to the pack pickup slot 17 after the first one of the maximum number of medicine packages 2 that can be stored in the casing 11 is picked up.

The pack posture keeper 15 keeps the posture of the medicine package 2.

The right support portion 12 and the left support portion 13 also support or hold the medicine package 2 in the casing 11.

In the present embodiment, the to-be-picked-up portion of one of the multiple medicine packages 2 to be picked up from one of the multiple cartridges 10 by the pickup unit 50 is at a lower portion or bottom portion of the corresponding one of the multiple cartridges 10. In other words, the to-be-picked-up portion includes the pack pickup slot 17, and the right support portion 12 and the left support portion 13 that serve as a pair of support portions or supporting member and support, at a plurality of points, one of the multiple medicine packages 2 to be picked up from one of the multiple cartridges 10.

The right support portion 12 and the left support portion 13 according to the present embodiment are configured to allow one of the multiple medicine packages 2 to pass through when one of the multiple medicine packages 2 is picked up from one of the multiple cartridges 10 by the pickup unit 50. On the other hand, when any one of the multiple medicine packages 2 is not to be picked up from one of the multiple cartridges 10, the multiple medicine packages 2 are to be stored and held in the casing 11. In order to achieve that, the right support portion 12 and the left support portion 13 are configured to restrict the passage of one of the multiple medicine packages 2

As described above, the right support portion 12 and the left support portion 13 according to the present embodiment serve as a pair of support portions that support or hold the multiple medicine packages 2 in the one of the multiple cartridges 10, and each one of the right support portion 12 and the left support portion 13 is fixed and immovable such that one of the multiple medicine packages 2 can be picked up in a stable manner from one of the multiple cartridges 10 by the pickup unit 50.

The right support portion 12 and the left support portion 13 according to the present embodiment are a pair of fixation members that are fixed or attached to the pair of bottom-wall inner surfaces 11e of the right bottom-wall edge and left bottom-wall edge of the pack pickup slot 17, respectively.

The pack pickup slot 17 according to the present embodiment has both a function to allow a pair of suction pads 52 of the pickup unit 50 illustrated in FIG. 4B to pass therethrough in order to pick up one of the multiple medicine packages 2 and a function to allow the picked-up medicine package 2 and the pair of suction pads 52 to pass therethrough. In other words, the pack pickup slot 17 and the above right bottom-wall edge and left bottom-wall edge that are arranged so as to surround the pack pickup slot 17 together serve as a pack passing-through portion according to the present embodiment.

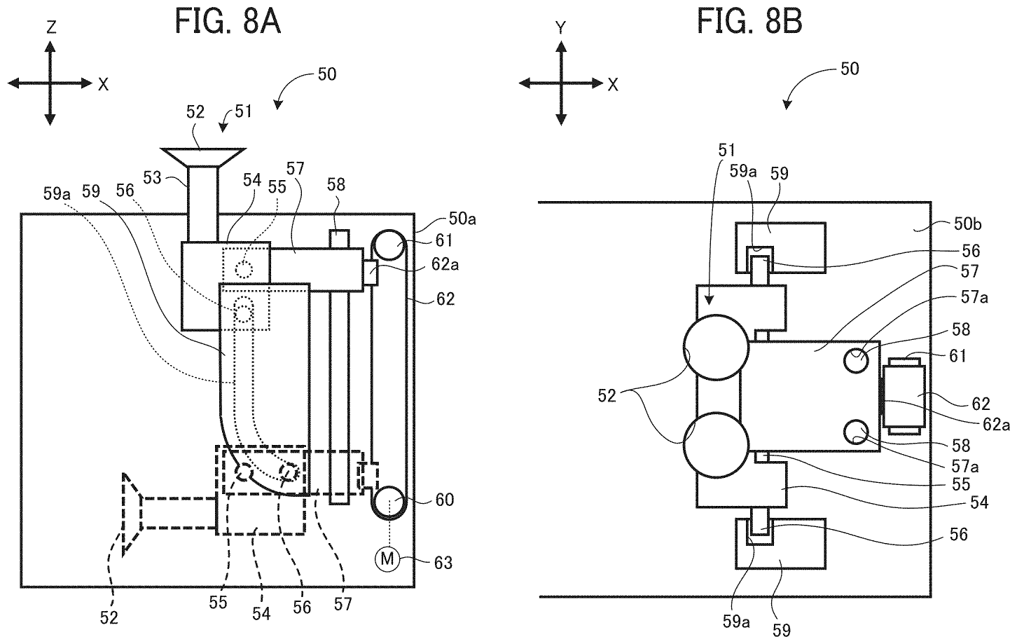

In one the multiple cartridges 10 illustrated in FIG. 4B, a pair of positions at which the pair of suction pads 52 adsorb or suck up one of the multiple medicine packages 2 stored in one of the multiple cartridges 10, as will be described later in detail with reference to FIG. 8A and FIG. 8B, are indicated by a pair of ring-shaped dot-dot-dash lines. Such a pair of positions may be referred to as a pair of suction pad positions in the following description.

The right support portion 12 and the left support portion 13 support the multiple medicine packages 2 in one of the multiple cartridges 10 such that the multiple medicine packages 2 in one of the multiple cartridges 10 do not fall from the pack pickup slot 17. As will be described later in detail in regard to the operation of the pickup unit 50, the right support portion 12 and the left support portion 13 have a positional relation in which one of the multiple medicine packages 2 is sucked by the pair of suction pads 52 at two suction pad positions in the Y-direction near both ends of the right support portion 12 when the lowermost one of the multiple medicine packages 2 in one of the multiple cartridges 10 is picked up while being sucked by the pair of suction pads 52. When the lowermost one of the multiple medicine packages 2 is picked up from one of the multiple cartridges 10 by the pair of suction pads 52, the pair of suction pads 52 passe near both ends of the right support portion 12 in the Y-direction to suck and hold one of the multiple medicine packages 2.

As illustrated in FIG. 4B, two points at which the suction pad 52 contacts are arranged near both ends of the right support portion 12 in the Y-direction. Due to such a configuration, the possibility of an error in which the suction by the pair of suction pads 52 is disabled can be avoided and prevented, and the picking up operation can successfully be done. In other words, as both ends of the multiple medicine packages 2 in the Y-direction are sucked by the pair of suction pads 52, the bag 2a of the medicine package 2, which is made of film, is stretched and can withstand the deformation. Accordingly, both reliable support or holding of the multiple medicine packages 2 in one of the multiple cartridges 10 and smooth removal of one of the multiple medicine packages 2 can be achieved.

The pack posture keeper 15 is formed of sponge rubber having appropriate elasticity. The movable board 16 is made of, for example, resin or metal. The pack posture keeper 15 and the movable board 16 according to the present embodiment hold the posture of the multiple medicine packages 2 under normal operating conditions in the casing 11. As explicitly illustrated in FIG. 4A, the posture of the multiple medicine packages 2 is held orderly in the Z-direction in a substantially horizontal state.

In order to achieve the above-described function, the movable board 16 is arranged to move at least one of the multiple medicine packages 2 left in the casing 11 toward the pack pickup slot 17 as moving downward in the casing 11 in the Z-axis direction by its own weight.

As illustrated in FIG. 4A, a long groove 11a that extends in the Z-direction with predetermined width in the X-direction is formed in the side wall of the casing 11. A shaft 16a with a flange is arranged at one end of the movable board 16 so as to protrude from the long groove 11a. As the shaft 16a of the movable board 16 is guided in the Z-direction parallel to the long groove 11a, the posture of the multiple medicine packages 2 can be held in the Z-direction. In FIG. 4, the multiple medicine packages 2 in the multiple cartridges 10 are stacked on top of each other in layers in a substantially horizontal state. However, in actuality, left portions of the medicine packages 2 in FIG. 4 filled with the medicines 3 are expanding. For a more realistic or actual state or condition in which a plurality of packs are stacked on top of each other and stored, see FIG. 5 as will be described later in detail.

A set of the multiple medicine packages 2 are orderly stored in the casing 11 toward the upper side from the pack pickup slot 17 on the right support portion 12 and the left support portion 13. The timing at which the multiple medicine packages 2 are supplied into one of the multiple cartridges 10 may be, for example, the timing at which a patient who takes medicines or occupant is examined in a day-care center or nursing home, which is typically once a two weeks, or the timing at which the multiple medicine packages 2 in one of the multiple cartridges 10 run out. When some medicine packages 2 are left in one of the multiple cartridges 10 at the time of replenishment, such replenishment is continued from the back of the remaining medicine packages 2.

Under normal operating conditions, the above-described setting of the multiple medicine packages 2 into one of the multiple cartridges 10 and the replenishment of the multiple medicine packages 2 are done by a staff or the like in a day-care center or nursing home. However, no limitation is indicated thereby. In particular, in configurations where cartridges are used instead of containers and setting or replenishment of the medicine packages 2 are done automatically, the setting or replenishment of the medicine packages 2 may be done differently.

The lid 14 is used by a staff or the like who works at a day-care center or nursing home to enable loading or unloading of the multiple medicine packages 2 stored in one of the multiple cartridges 10, and as illustrated in FIG. 4A, is formed with wide length in the Z-direction of the casing 11 and has a predetermined opening width.

As illustrated in FIG. 4A, the types of the multiple medicine packages 2 in one of the multiple cartridges 10 are sorted or divided based on the timings at which medicines are taken. For example, one cartridge contains medicines to be taken in fourteen days in the morning by Mr. or Ms. A. In view of the above circumstances, if Mr. or Ms. A takes the medicines not only in the morning but also in the daytime or in the evening, or before going to bed, four cartridges 10 are required in total.

However, no limitation is indicated thereby. For example, in an alternative embodiment of the present disclosure, a single cartridge 10 may be prepared for each person or patient who takes medicines. In such cases, for example, a pack to be used in the morning on day one, a pack to be used at lunchtime on day one, a pack to be used in the evening on day one, a pack to be used before going to bed on day one, a pack to be used in the morning on day two, a pack to be used at lunchtime on day two, and a pack to be used in the evening on day two may be stored in the order listed upward from the pack pickup slot 17 parallel to the direction in which packs are taken out from the multiple cartridges 10.

Some technologies have been proposed for a combination between a left flap portion and a right flap portion of the flap mechanism. Such a left flap portion is provided with a helical torsion coil spring having pressing force within a predetermined range between the rotation axis and the right bottom-wall edge, and such a right flap portion is openable and closable and can swing about a rotation axis arranged around the right bottom-wall edge of the pack pickup slot 17. However, in such proposed technologies, there are technical problems in picking up a pack from the cartridge 10 or the storage unit in view of the results of some specific experiments.

In order to deal with the above technical problems, in some embodiments of the present disclosure, the right support portion 12 and the left support portion 13 are fixed to the bottom-wall inner surface 11e of the pack pickup slot 17 of the casing 11 such that one of the multiple medicine packages 2 can be picked up in a steadily stable manner from one of the multiple cartridges 10 by the pickup unit 50.

In other words, the right support portion 12 and the left support portion 13 according to the present embodiment are fixed when the right support portion 12 and the left support portion 13 hold both ends of the medicine pack such as the medicine package 2 or the bound packages 2A around the pack pickup slot 17 arranged at the lower portion of one of the multiple cartridges 10.

An end of the bottom one of the multiple medicine packages 2 to be adsorbed by, for example, an adsorption system is supported by the right support portion 12, and the other end of the bottom one of the multiple medicine packages 2 is supported by the left support portion 13. Due to such a configuration, the medicine packages 2 that are set and stored in the cartridge 10 do not drop.

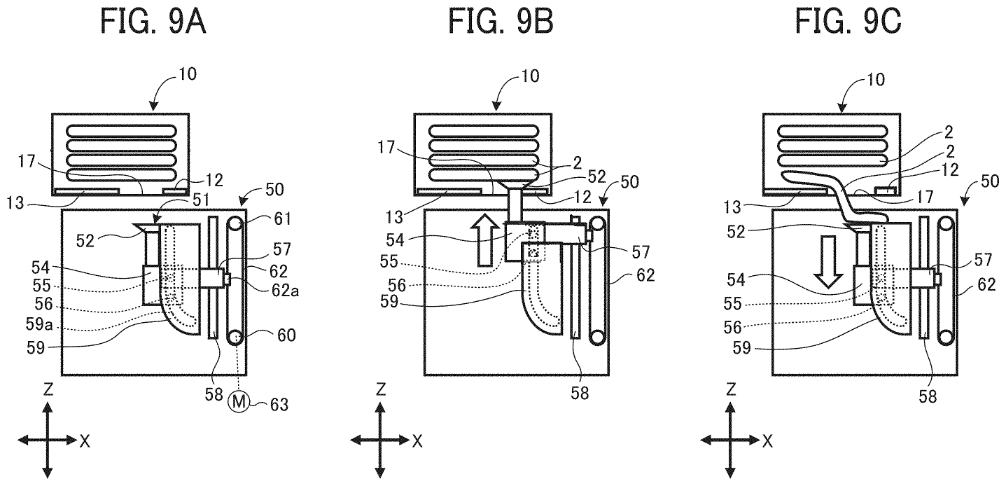

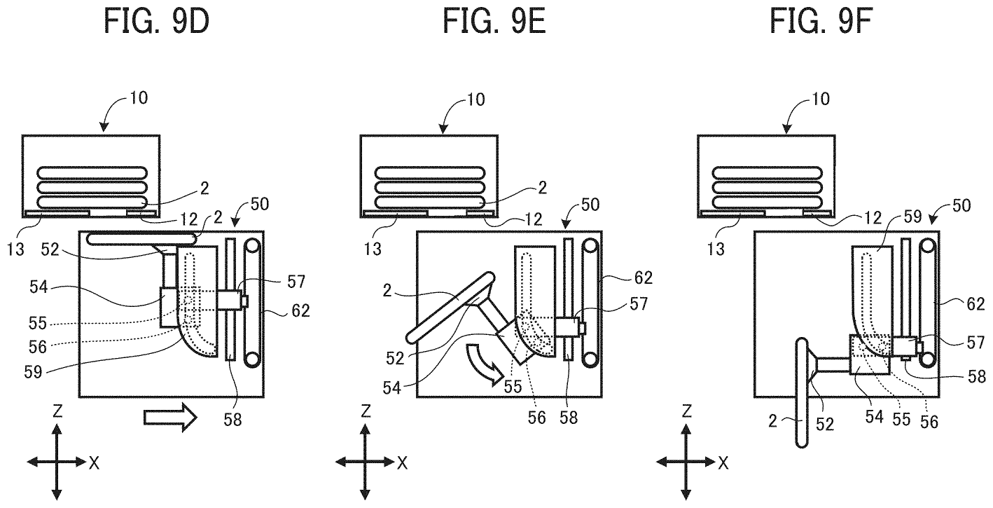

The right support portion 12 and the left support portion 13 are different in the length of the portion that support the multiple medicine packages 2, and the length of portion of the right support portion 12 that support the multiple medicine packages 2 is shorter than that of the left support portion 13. As will be described later in detail with reference to FIG. 9C, when the lowermost one of the multiple medicine packages 2 stored in the cartridge 10 is adsorbed or sucked by the pair of suction pads 52 and is pulled out from the cartridge 10, the medicine pack is bent due to its elasticity. Accordingly, the medicine pack can easily be pulled out.

As the pair of support portions including the right support portion 12 and the left support portion 13 are of fixed type, the front end of the next one of the multiple medicine packages 2 to be taken out can be reliably held and does not jump out or fall off together with one of the multiple medicine packages 2 that is being taken out. As the pair of support portions including the right support portion 12 and the left support portion 13 do not swing or rotate, the medicine package 2 are held with high stability with no deformation or the like due to unintentional nipping or pressing in the returning operation.

The medicine pack and the cartridge 10 are described with reference to FIG. 5 in a further magnified and exaggerated manner than the medicine pack stored in the cartridge 10 of FIG. 4A. The bottom view of the cartridge 10 of FIG. 5 is equivalent to that of FIG. 4B.

In FIG. 5, each one of the multiple medicine packs such as the multiple medicine packages 2 or the bound packages 2A that are stored in one of the multiple cartridges 10 is sealed on the right support portion 12 side. Accordingly, in FIG. 5, the left portion of each one of the multiple medicine packs near the left support portion 13 has an expanding shape due to the medicines inside. When the right sides of the packs are bound by a stapler or the like as in the bound packages 2A (see FIG. 2C), the left portions of the packs further expand.

In order to deal with such a situation, as illustrated in FIG. 5, the height and thickness of the right support portion 12 are increased to be greater than the height and thickness of the left support portion 13 for the support of the medicine packs. Due to such a configuration, the held posture of the medicine packs in one of the multiple cartridges 10 does not incline in an undesired manner, and the sucking operation can be stably performed.

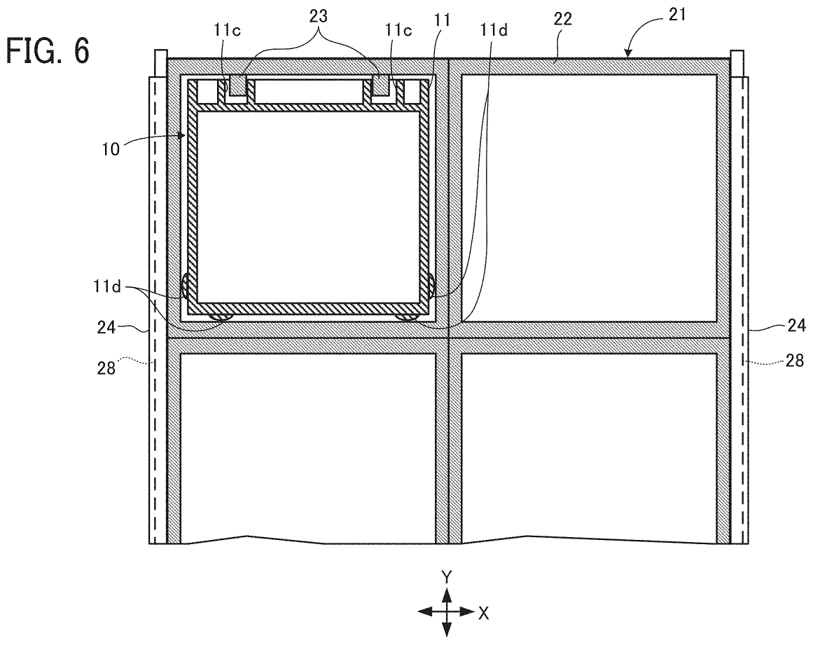

FIG. 6 is a schematic plan sectional view of the attaching and detaching mechanisms for a container, which are arranged on the drawer 21, according to the present embodiment.

FIG. 7 is a schematic plan view of the drawer illustrating the mechanisms for recognizing a container, which are arranged on the drawer 21, according to the present embodiment.

As illustrated in FIG. 6, a plurality of cartridges 10 can be attached to or detached from the drawer 21. The casing 22 of the drawer 21 is provided with a pair of slide rails 24 on the right and left outer side walls, respectively, and each one of the pair of slide rails 24 is slidable with one of the multiple rails 28 provided for the main structural frame 199 (see FIG. 1A and FIG. 1B). Due to such a configuration, the drawer 21 can be attached or detached by being pulled out from the main structural frame 199 (see FIG. 1A and FIG. 1B) through the engagement between the slide rail 24 and the one of the multiple rails 28 of the main structural frame 199.

As illustrated in FIG. 6, the cartridge 10 is attached to and detached from the drawer 21 through the engagement and disengagement between a pair of concave portions 11c on the external wall of the casing 11 of the cartridge 10 and a pair of convex portions 23 that are formed on the inner wall of the casing 22 of the drawer 21 and protrude inwardly and the engagement and disengagement between the inner wall of the casing 22 and four hemispherical protrusions 11d on the external wall of the casing 11. With the above-described attaching and detaching mechanism of the drawer 21, the multiple cartridges 10 can be attached or detached easily and with good operability.

In the above embodiments of the present disclosure, attachment and detachment are enabled by the coupling or engagement of concave and convex portions. However, no limitation is intended thereby, and an elastic member may be arranged in the gap between the inner wall of the casing 22 and the external wall of the casing 11 to enable attachment and detachment. Alternatively, a configuration or structure using magnetic force or a snap-fit assembly method may enable attachment and detachment.

As illustrated in FIG. 7, the drawer 21 according to the present embodiment is provided with an information display device such as a plurality of light-emitting diodes (LEDs) 25a, 25b, 25c, 25d, 25e, 25f, 25g, and 25h around a grip 26 that is held by hand to insert or withdraw the drawer 21. Due to such a configuration, the arrangement of the multiple cartridges 10 can easily be figured out. More specifically, due to such a configuration, at what position the cartridge 10 in focus is placed in the drawer 21 can easily be figured out at a glance. In FIG. 6, the light-emitting diode (LED) 25a is used to detect the presence or absence of one of the multiple cartridges 10 attached to or detached from A1 of the drawer 21. A1 denotes a position or partition that is uniquely determined in the vertical columns and horizontal rows. In a similar manner to the above, the LED 25b, the LED 25c, the LED 25d, the LED 25e, the LED 25f, the LED 25g, and the LED 25h detect the presence or absence of one of the multiple cartridges 10 to be attached to or detached from A2, A3, A4, B1, B2, B3, and B4 of the drawer 21, respectively.

With the use of an information display device such as the LED 25a, LED 25b, the LED 25c, the LED 25d, the LED 25e, the LED 25f, the LED 25g, and the LED 25h, there is a possibility that a staff or the like who is an operator and attaches or detaches one of the multiple cartridges 10 mix up which one to attach or detach. In order to avoid such a situation, for example, the presence or absence of the cartridges 10 may be electronically determined by, for example, a sensor or a switch.

The container may be provided with for example, a number, a bar code, a quick response (QR) code (registered trademark), or a non-contact integrated circuit (IC) tag such that each one of the containers will be identifiable. It is desired that the system be programmed to remember what container stores whose medicines. Subsequently, the drawer to which the multiple containers are attached is set in the main structural frame 199 of the medication support device 200, and each one of the multiple containers is identified by the medication support device 200. Due to such a configuration, a desired pack can be picked up in the medication support device 200 without causing any errors.

FIG. 8A is a front view of the pickup unit 50, illustrating a configuration or structure of the pickup unit 50, according to the present embodiment.

FIG. 8B is a plan view of the pickup unit 50 illustrated in FIG. 8A, according to the present embodiment. FIG. 9A, FIG. 9B, FIG. 9C, FIG. 9D, FIG. 9E, and FIG. 9F are front views the pickup unit 50 illustrating the progression of the operation of the pickup unit 50, according to the present embodiment.

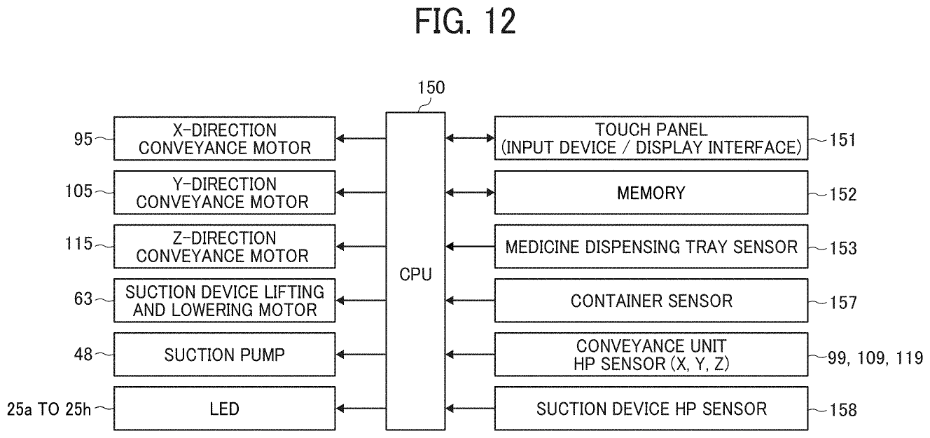

As illustrated in FIG. 8A and FIG. 8B, the pickup unit 50 according to the present embodiment includes a suction device 51 that picks up and holds one of the multiple medicine packages 2 from one of the multiple cartridges 10. The suction device 51 has a suction pump 48 using air (see the block diagram of FIG. 12 as will be described later in detail), and the suction pump 48 places the suction device 51 under negative pressure. By so doing, one of the multiple medicine packages 2 is successfully sucked up.

The suction pump 48 may be arranged in the pickup unit 50 or may be disposed at another portion in the medication support device 200. When the suction pump 48 is arranged inside the medication support device 200, the suction device 51 is connected to the suction pump 48 through a communicating member such as an air tube.

The suction device 51 has a suction pad 52 that communicates with the suction pump 48 and sucks one of the multiple medicine packages 2, and a suction duct 53 coupled to the pair of suction pads 52. The suction pad 52 according to the present embodiment serves as a suction unit or a suction member that sucks up and picks up one of the multiple medicine packages 2 in one of the multiple cartridges 10.

As described above, the upper ends of the pair of suction pads 52 as illustrated in FIG. 8A is arranged to absorb one of the multiple medicine packages 2. The bottom ends of the pair of suction pads 52 as illustrated in FIG. 8A are attached and fixed to the upper end of the suction duct 53 as illustrated in FIG. 8A. The bottom end of the suction duct 53 as illustrated in FIG. 8A is attached and fixed to the rotation base 54. A pair of suction pads 52 and suction ducts 53 are arranged in the Y-direction.

The pickup unit 50 according to the present embodiment is provided with a posture changing unit used to change the posture of one of the multiple medicine packages 2 picked up from one of the multiple cartridges 10 to a substantially vertical posture. The posture changing unit in the pickup unit 50 may include, for example, a rotation base 54 coupled to a fixation member 57 through a rotation axis 55, a guide unit 59 formed with a guide groove 59a having a specific shape, a guide axis 56 that is consistently fitted into the guide groove 59a of the guide unit 59 to guide the rotation base 54, and a suction unit lifting and lowering unit.

The rotation base 54 is coupled to the fixation member 57 through the rotation axis 55. The rotation base 54 may be may rotatably or pivotably be arranged around the rotation axis 55 fixed to the rotation base 54 within a range of a predetermined angle. Alternatively, the rotation base 54 may be may rotatably or pivotably be arranged around the rotation axis 55 fixed to the fixation member 57. In other words, in FIG. 8A and FIG. 8B, the distance between the center of the rotation axis 55 and the center of a guide rod 58 in the X-direction is kept constant at all times when the fixation member 57 moves along the guide rod 58 in the up-and-down directions parallel to the Z-direction. The guide rod 58 will be described later in detail.

The suction pad lifting and lowering unit includes a pair of guide rods 58 that are arranged separately in the Y-direction and guides the fixation member 57 in the Z-direction, an endless belt 62 looped around a driving pulley 60 and a driven pulley 61, and a suction device lifting and lowering motor 63 coupled to the driving pulley 60 through a driving power conveyor such a gear or a belt. The suction device lifting and lowering motor 63 according to the present embodiment serves as a driver circuit or a driving source of the suction pad lifting and lowering unit.

The fixation member 57 according to the present embodiment is coupled and fixed to the endless belt 62 at a belt grip 62a fixed to the right end of the fixation member 57.

The pair of guide rods 58 are arranged at two different positions in the Y-direction, and extend in the Z-direction. The bottom ends of those guide rods 58 are fixed to the base frame 50b of the picking-up frame 50a provided for the pickup unit 50.

A pair of guiding holes 57a into which the pair of guide rods 58 are inserted are formed around a right end of the fixation member 57.

The pulley axis of each one of the driving pulley 60 and the driven pulley 61 is rotatably supported by an immovable member on the picking-up frame 50a. The suction device lifting and lowering motor 63 is fixed to an immovable member provided for the picking-up frame 50a of the pickup unit 50. The suction device lifting and lowering motor 63 is also a driver circuit to be controlled (see FIG. 12 as will be described later in detail) of the suction unit lifting and lowering unit.

When the fixation member 57 is lifted or lowered by the operation of the suction device lifting and lowering motor 63, the fixation member 57 is moved along the pair of guide rods 58 in the Z-direction. Accordingly, the posture of the fixation member 57 on the XY plane can be kept constant in a substantially horizontal state.

The suction unit lifting and lowering unit is not limited to the above-described up-and-down reciprocating mechanism driven by a belt. Alternatively, for example, the suction unit lifting and lowering unit may have a reciprocating linear motion mechanism using a rack and pinion.

A pair of guide units 59 are arranged on both sides of the suction device 51 in the Y-direction with the rotation base 54 interposed therebetween, and the bottom ends of the pair of guide units 59 are fixed to the base frame 50b.

The guide axis 56 is arranged so as to protrude from both ends of the rotation base 54 in the Y-direction, and is consistently fitted into the guide groove 59a of the guide unit 59 to guide the rotation base 54. As illustrated in FIG. 8A, the guide axis 56 is provided below the rotation axis 55 of the rotation base 54 in the Z-direction with a certain distance from the rotation axis 55.

When the fixation member 57 is moved in the Z-direction by the operation of the suction device lifting and lowering motor 63, the guide axis 56 of the rotation base 54 is moved in the Z-direction parallel to the guide groove 59a having a specific shape while maintaining the posture of the fixation member 57 on the XY plane constant in a substantially horizontal state. Due to such a configuration, the posture of the suction pad 52 can be rotated by approximately 90 degrees. In FIG. 8A, the suction device 51 that is rotated by approximately 90 degrees is indicated by thick broken lines. In the present embodiment, the expression “substantially horizontal state” indicates that the posture of a particular element is within a specific range of tolerance for angle with respect to the horizontal, which is allowable in achieving the effects of the embodiments of the present disclosure, in addition to a state in which the position of a particular element is horizontal.

The guide groove 59a having a specific shape includes the first guide groove portion that extends in the Z-direction with relatively great length so as to hold the posture of the pair of suction pads 52 upward as indicated by solid lines in FIG. 8A through the rotation base 54 in a substantially horizontal state as guided by the guide axis 56, and the second guide groove portion that gently draw an obtuse and shallow arc to the right side as it goes downward to rotate the posture of the rotation base 54 and the pair of suction pads 52 by approximately 90 degrees. The first guide groove portion and the second guide groove portion communicate with each other and are coupled to each other.

FIG. 9A, FIG. 9B, FIG. 9C, FIG. 9D, FIG. 9E, and FIG. 9F are front views a pickup unit 50 illustrating the progression of the operation of the pickup unit 50, according to the present embodiment.

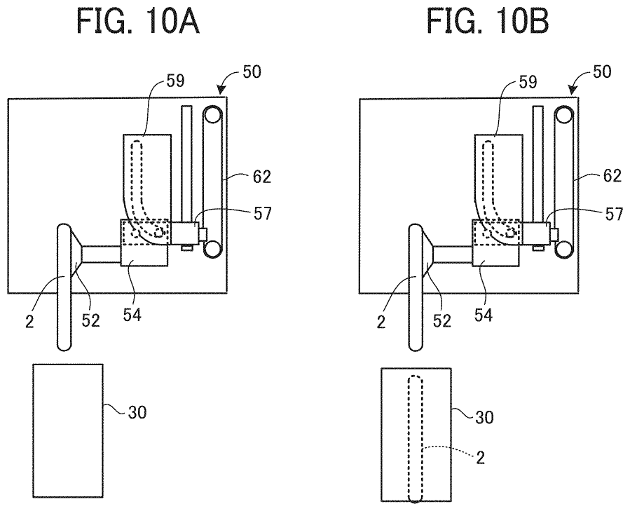

FIG. 10A and FIG. 10B are front views of the pickup unit 50 illustrating the progression of the operation of the pickup unit 50 subsequent to the progression illustrated in FIG. 9A, FIG. 9B, FIG. 9C, FIG. 9D, FIG. 9E, and FIG. 9F, according to the present embodiment.

For the sake of explanatory convenience, it is assumed in the present embodiment that, by the operation of the conveyance unit 90 illustrated in FIG. 1A and FIG. 1B, the pickup unit 50 is arranged between the multiple cartridges 10 of the drawer 21 arranged at the uppermost portion of the main structural frame 199 in FIG. 1A and FIG. 1B and the multiple medicine dispensing trays 30 disposed directly below the multiple cartridges 10. In the present embodiment, it is assumed that the cartridges illustrated in FIG. 4A and FIG. 4B are used as the multiple cartridges 10. As illustrated in FIG. 9A, the pickup unit 50 is moved under one of the multiple cartridges 10 by the operation made by the conveyance unit 90 illustrated in FIG. 1A and FIG. 1B, and stops moving. Under such conditions, the suction device lifting and lowering motor 63 of the suction unit lifting and lowering unit is stopped, and the pair of suction pads 52 are positioned under the position of the top face of the pickup unit, which indicates the position of the top face of the picking-up frame 50a of the pickup unit 50 in a housing. Subsequently, as illustrated in FIG. 9B, the pair of suction pads 52 are moved upward by the operation of the suction device lifting and lowering motor 63, and enters from the pack pickup slot 17 between the right support portion 12 and the left support portion 13. Then, as soon as the pair of suction pads 52 contact one of the multiple medicine packages 2 at the bottom of one of the multiple cartridges 10, and the pair of suction pads 52 suck that medicine package 2. In so doing, the suction pump 48 as described above is driven to operate in advance so that the suctioning operation can be performed.

Subsequently, as illustrated in FIG. 9C, as the suction device lifting and lowering motor 63 is reversely operated, the pair of suction pads 52 move downward while sucking one of the multiple medicine packages 2 by the pair of suction pads 52, and the front end of one of the multiple medicine packages 2 is pulled out from one of the multiple cartridges 10. The front end of the medicine package indicates a side of the medicine package to be absorbed or sucked up by the pair of suction pads 52, and the same applies in the following description. The operation of pulling out the front end of one of the multiple medicine packages 2 from the pack pickup slot 17 of one of the multiple cartridges 10 results in sort of free deformation in the pulled-out medicine package 2.

Subsequently, as illustrated in FIG. 9D, by the operation of the conveyance unit 90 (see FIG. 1A and FIG. 1B), the pickup unit 50 is moved in the X-direction, which is the lateral direction, and the rear end of one of the multiple medicine packages 2 is drawn out or picked up from one of the multiple cartridges 10. Immediately after that, as illustrated in FIG. 9E and FIG. 9F, by the operation of the suction device lifting and lowering motor 63, one of the multiple medicine packages 2, which is approximately in a horizontal state and is sucked and held by the pair of suction pads 52, is rotated by substantially 90 degrees to change the posture to an approximately vertical or upright posture. In such rotational operation, the rotation axis 55 that is arranged on the rotation base 54 moves along the guide groove 59a of the guide unit 59. Accordingly, the posture of one of the multiple medicine packages 2 can be changed from an approximately horizontal posture to an approximately vertical posture. The above series of operation in the above configuration may be achieved by a series of operation performed by a single suction device lifting and lowering motor 63.

Subsequently, as illustrated in FIG. 10A, once the pickup unit 50 that includes the pair of suction pads 52 and holds one of the multiple medicine packages 2 in a substantially vertical posture is carried by the conveyance unit 90 to a position substantially directly above the position where multiple medicine dispensing trays 30 are arranged, the operation of the above suction pump 48 is terminated. As a result, the suction holding of one of the multiple medicine packages 2 by the pair of suction pads 52 is released, and one of the multiple medicine packages 2 is inserted into one of the multiple partitions 33 that is a predetermined position in one of the multiple medicine dispensing trays 30 (see FIG. 10B).

After the above-described operation is performed a plurality of times and a desired one of the multiple medicine packages 2 is inserted into one of the multiple partitions 33 that is at a predetermined position of one of the multiple medicine dispensing trays 30, one of the multiple medicine dispensing trays 30 is ejected to the outside of the medication support device 200 through the third gate 43, and is received by a staff or the like in a day-care center or nursing home or a medication assistant.

As described above, the pickup unit 50 takes out the front end of the pack to a position under one of the multiple cartridges 10 in the process of picking up out one of the multiple medicine packages 2 from one of the multiple cartridges 10, and the conveyance unit 90 according to the present embodiment moves the pickup unit 50 so that the rear end of one of the multiple medicine packages 2 is picked up from one of the multiple cartridges 10 when the front end of one of the multiple medicine packages 2 has taken out to a position under one of the multiple cartridges 10. The moving direction of the pickup unit 50 by the conveyance unit 90 is set to the X-direction parallel to the lateral direction in which the rear end of one of the multiple medicine packages 2 is released from the support of the left support portion 13 and is drawn out from the pack pickup slot 17.

In the above embodiments of the present disclosure, when desired one of the multiple medicine packages 2 is picked up from one of the multiple cartridges 10, the pickup unit 50 is positioned or arranged under one of the multiple cartridges 10, and such desired one of the multiple medicine packages 2 is taken out in the downward direction of one of the multiple cartridges 10. When one of the multiple medicine packages 2 is taken out from the lower side of one of the multiple cartridges 10 as described above, the next one of the multiple medicine packages 2 automatically moves downward or toward the pack pickup slot 17 due to the self weight of the movable board 16 and the multiple medicine packages 2 left in the cartridge 10. Due to such a configuration, the pickup unit 50 can perform the same operation with a relatively simple configuration regardless of the number of the multiple medicine packages 2 left in the cartridge 10.

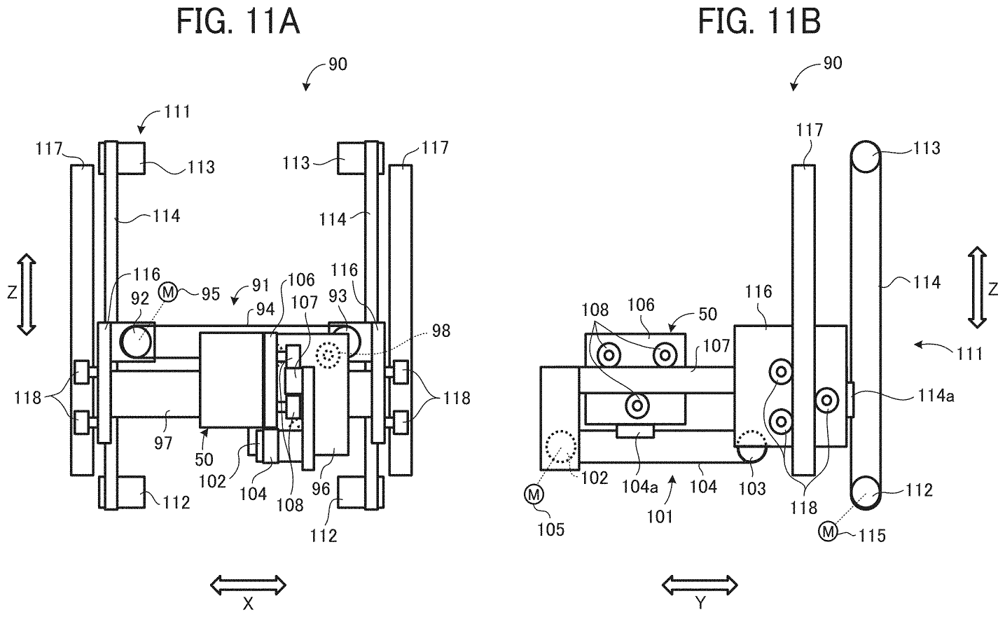

The configuration and operation of the conveyance unit 90 is described below with reference to FIG. 11A and FIG. 11B.

FIG. 11A is a front view of the conveyance unit 90 illustrating a schematic configuration of the conveyance unit 90, according to the present embodiment.

FIG. 11B is a side view of the conveyance unit 90 illustrated in FIG. 11A, according to the present embodiment.

As in the configuration or structure of the medication support device 200 illustrated in FIG. 1A and FIG. 1B, the multiple cartridges 10 are aligned in a row on a pair of planes above and below the multiple medicine dispensing trays 30 in the Z-direction, the multiple medicine dispensing trays 30 are arranged above the multiple cartridges 10 on the lowermost one of the above pair of planes. In view of these circumstances, the pickup unit 50 is configured to move in three directions of the X-direction, the Y-direction, and the Z-direction. As described above, the conveyance unit 90 moves the pickup unit 50 in the X-direction, in the Y-direction, and in the Z-direction in order to convey one of the multiple medicine packages 2 picked up from one of the multiple cartridges 10 by the pickup unit 50 and pass it to one of the multiple medicine dispensing trays 30.

The pickup unit 50 is moved in the X-direction by an X-direction conveyance unit 91, and the pickup unit 50 is moved in the Y-direction by a Y-direction conveyance unit 101. The pickup unit 50 is moved in the Z-direction by a Z-direction conveyance unit 111. These three conveyance units have a similar configuration or structure.

The X-direction conveyance unit 91 according to the present embodiment includes a X-adaptor 96 attached to the pickup unit 50, a X-guide unit 97 that guides the pickup unit 50 in the X-direction through the X-adaptor 96, an endless belt 94 looped around a driving pulley 92 and a driven pulley 93, and a X-direction conveyance motor 95 coupled to the driving pulley 92 through a driving force conveyor such as a gear or a belt.

As illustrated in FIG. 11B, three rollers 98 are attached to the X-adaptor 96 so as to clamp the X-guide unit 97. Due to such a configuration, the X-adaptor 96 can roll over the X-guide unit 97. Note that, in FIG. 11A, two of the three rollers 98 are hidden by the pickup unit 50. The X-adaptor 96 is fixedly coupled to the endless belt 94 through a belt grip.

With the above-described configuration of the X-direction conveyance unit 91, as the X-direction conveyance motor 95 is driven, the driving force is conveyed to the endless belt 94 through the driving force conveyor and the driving pulley 92. Accordingly, the endless belt 94 rotates, and the pickup unit 50 moves in the X-direction parallel to the X-guide unit 97 together with the X-adaptor 96.

The Y-direction conveyance unit 101 according to the present embodiment includes a Y-adaptor 106 attached to the pickup unit 50, a Y-guide unit 107 that guides the pickup unit 50 in the Y-direction through the Y-adaptor 106, an endless belt 104 looped around a driving pulley 102 and a driven pulley 103, and a Y-direction conveyance motor 105 coupled to the driving pulley 102 through a driving force conveyor such as a gear or a belt.

As illustrated in FIG. 11B, three rollers 108 are attached to the Y-adaptor 106 so as to clamp the Y-guide unit 107. Due to such a configuration, the Y-adaptor 106 can roll over the Y-guide unit 107. The Y-adaptor 106 is coupled and fixed to the endless belt 104 through the belt grip 104a.

With the above-described configuration of the Y-direction conveyance unit 101, as the Y-direction conveyance motor 105 is driven, the driving force is conveyed to the endless belt 104 through the driving force conveyor and the driving pulley 102. Accordingly, the endless belt 104 rotates, and the pickup unit 50 moves in the Y-direction parallel to the Y-guide unit 107 together with the Y-adaptor 106.

The Z-direction conveyance unit 111 includes a pair of Z-adaptors 116 attached to both ends of the X-guide unit 97 in the X-direction, a pair of Z-guide units 117 that guide the pickup unit 50 in the Z-direction through the X-guide unit 97 and the pair of Z-adaptors 116, a pair of endless belts 114 each of which is looped around a drive pulley 112 and a driven pulley 113, and a Z-direction conveyance motor 115 coupled to the pair of drive pulley 112 through a driving force conveyor such as a gear or a belt.

In the Z-direction conveyance unit 111, the pair of driving pulleys 112, the pair of driven pulleys 113, and the pair of endless belts 114 are arranged on both sides in the X-direction, respectively. By contrast, the Z-direction conveyance motor 115 is provided for only on one of the pair of driving pulleys 112.

As illustrated in FIG. 11B, three rollers 118 are attached to each one of the pair of Z-adaptors 116 so as to clamp corresponding one of the pair of Z-guide units 117. Due to such a configuration, the pair of Z-adaptors 116 can roll over the pair of Z-guide units 117, respectively. The pair of Z-adaptors 116 are coupled and fixed to the pair of endless belts 114 through a pair of belt grips 114a, respectively.

With the above-described configuration of the Z-direction conveyance unit 111, as the Z-direction conveyance motor 115 is driven, the driving force is conveyed to the pair of endless belts 114 through the driving force conveyor and the driving pulley 112. Accordingly, the pair of endless belts 114 rotates, and the pickup unit 50 moves in the Z-direction parallel to the Z-guide unit 117 together with the X-guide unit 97 and the Z-adaptor 116.

In FIG. 11A and FIG. 11B, the pickup unit 50 is configured to move in the triaxial directions including the X-axis direction, the Y-axis direction, and the Z-axis direction. However, no limitation is indicated thereby. For example, when the multiple cartridges 10 are arranged above the pickup unit 50 and the multiple medicine dispensing trays 30 are arranged below the pickup unit 50, the pickup unit 50 only needs to move in the X-direction and the Y-direction. In such cases, the number of axes of motion can be reduced by one.

FIG. 12 is a control block diagram illustrating a schematic control structure for the medication support device 200 according to the present embodiment.

As illustrated in FIG. 12, the medication support device 200 includes a central processing unit (CPU) that serves as a controller 150 that controls the operation of, for example, the components or elements of the medication support device 200. For example, the CPU may be provided with a built-in memory or a built-in timer. The CPU according to the present embodiment may provide notification to a staff or the like at a timing consistent with the program or may instruct the medication support device 200 to perform particular operation, based on various kinds of input such as the inputs from a sensor as will be described later in detail.

The CPU may have, for example, a computing or control function, and a timer or clocking function. A memory 152 includes, for example, a read-only memory (ROM), a random access memory (RAM), and an external memory. The ROM stores, for example, a program readable by the above CPU and various kinds of data in advance. Such a program stored in the ROM may be a program used in the flowchart of the controlling processes as will be described later in detail. The above various kinds of data may be, for example, the data about the relation between the multiple medicine packages 2 and the multiple partitions 33 of one of the multiple medicine dispensing trays 30 allocated to each of the patients who take medicines, the data about the relation between the multiple medicine packages 2 and the multiple partitions 33 of one of the multiple medicine dispensing trays 30 assigned to each one of the times of medication, or the data about the relation between the multiple medicine packages 2 and the multiple partitions 33 of one of the multiple medicine dispensing trays 30 sorted according to the order in which medicines are to be taken.

The CPU according to the present embodiment has an input and output (I/O) port, and a touch panel 151 that serves as a user interface (UI) is electrically connected to that input and output port of the CPU. However, no limitation is intended thereby, and the touch panel 151 may be, for example, a combination of an input device and a display interface that are arranged separately. For example, a combination of a keyboard and a light-emitting diode (LED) display may be used as the touch panel 151.

The CPU according to the present embodiment has an input port, and a medicine dispensing tray sensor 153 that detects the type of medicine dispensing tray 30 stored in the medication support device 200 or determines whether or not there is any medicine dispensing tray 30, and a container sensor 157 that determines whether or not there is any cartridge 10 are electrically connected to the input port of the CPU.

Moreover, to the input port of the CPU, a conveyance unit home position (HP) sensor 99 that detects the home position (HP) of the X-direction conveyance unit 91 in the pickup unit 50, a conveyance unit home position (HP) sensor 109 that detects the home position (HP) of the Y-direction conveyance unit 101 in the pickup unit 50, and a conveyance unit home position (HP) sensor 119 that detects the home position (HP) of the Z-direction conveyance unit 111 in the pickup unit 50 are electrically connected. Note that such a home position (HP) of each conveyance unit may be abbreviated to HP in the following description.

Further, to the input port of the CPU, a suction device home position (HP) sensor 158 that detects the home position (HP) of the suction pad 52 of the suction device 51 in the pickup unit 50 is electrically connected.

To the output port of the CPU, the LED 25a, the LED 25b, the LED 25c, the LED 25d, the LED 25e, the LED 25f, the LED 25g, and the LED 25h of the drawer 21, the suction pump 48, the suction device lifting and lowering motor 63, the X-direction conveyance motor 95 of the X-direction conveyance unit 91, the Y-direction conveyance motor 105 of the Y-direction conveyance unit 101, and the Z-direction conveyance motor 115 of the Z-direction conveyance unit 111 are electrically connected.

To the output port of the CPU, a notification unit may be electrically connected. Such a notification unit reports what sort of state or conditions the components or elements of the medication support device 200 are in by means of, for example, the light emitted from a light-emitting diode (LED) and the sound or vibration including voice. Moreover, such a notification unit may be provided with, for example, a loudspeaker or a light that indicates that the medicines are to be taken so that the staff or the like away from the medication support device 200 can be notified of such a time of medication.

Once the input data from the touch panel 151 and various kinds of signals from various types of sensors are input to the CPU, a command signal is newly output from the CPU. In other words, the CPU outputs a command signal used to control the audio device or the optical device of the display device of the touch panel 151 including the above notification unit, the LED 25a, the LED 25b, the LED 25c, the LED 25d, the LED 25e, the LED 25f, the LED 25g, and the LED 25h, the suction pumps 48, the suction device lifting and lowering motor 63, the X-direction conveyance motor 95, the Y-direction conveyance motor 105, and the Z-direction conveyance motor 115.

The CPU according to the present embodiment has a function to execute the control operation as will be described later in detail in the following description or the flowchart of the controlling processes.

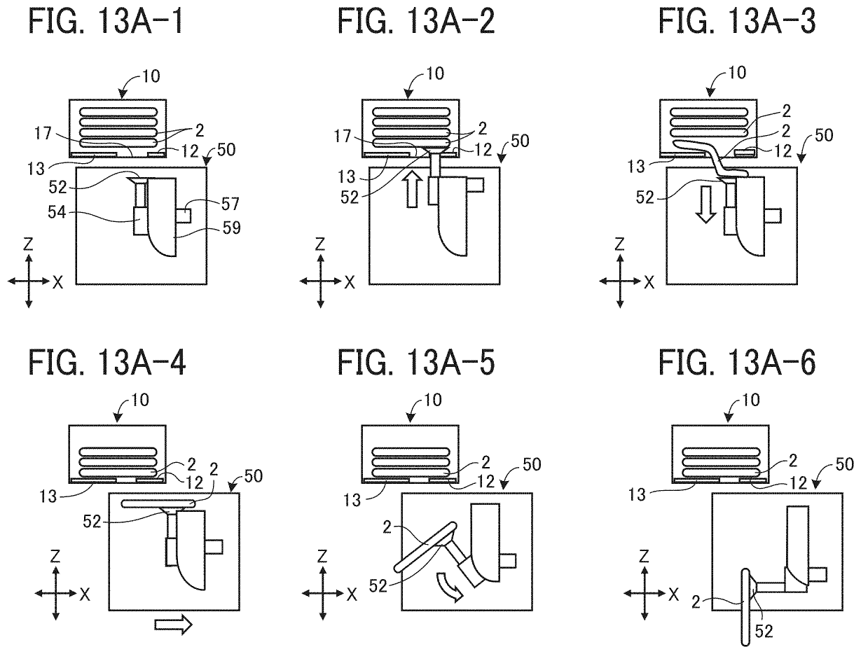

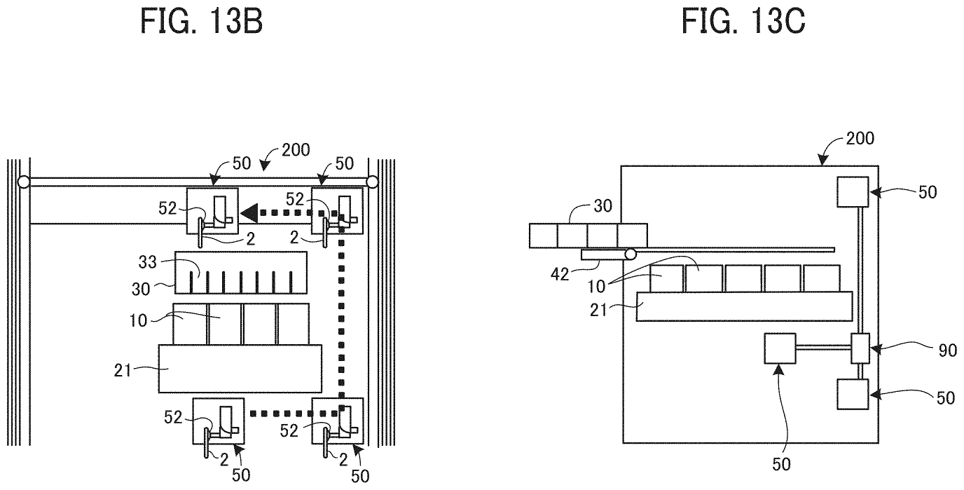

FIG. 13A-1, FIG. 13A-2, FIG. 13A-3, FIG. 13A-4, FIG. 13A-5, FIG. 13A-6, FIG. 13B, and FIG. 13C are schematic diagrams each illustrating an overall operation flow of the medication support device 200 illustrated in FIG. 1, according to the present embodiment.

The above operation is executed based on the control commands sent from the CPU of the controller 150.

The operation that is described above with reference to FIG. 9A, FIG. 9B, FIG. 9C, FIG. 9D, FIG. 9E, and FIG. 9F is further described in detail with reference to FIG. 13A-1, FIG. 13A-2, FIG. 13A-3, FIG. 13A-4, FIG. 13A-5, and FIG. 13A-6. The operating status illustrated in FIG. 13A-6 illustrates a state in which the posture of the pair of suction pads 52 sucking and holding one of the multiple medicine packages 2 picked up from one of the multiple cartridges 10 is rotated by approximately 90 degrees. As illustrated in FIG. 13B, the medicine package 2 that has been taken out is held by the pair of suction pads 52 of the pickup unit 50 in the same posture as that illustrated in FIG. 13A-6.

As illustrated in FIG. 13B, the pickup unit 50 that includes the pair of suction pads 52 and holds one of the multiple medicine packages 2 is conveyed by the conveyance unit 90 along the route indicated by thick broken lines to the medicine dispending position 29 where multiple medicine dispensing trays 30 are arranged. When the pickup unit 50 is carried to a position substantially directly above the multiple medicine dispensing trays 30 at the medicine dispending position 29, the operation of the suction pump 48 is terminated. As a result, the suction holding of one of the multiple medicine packages 2 by the pair of suction pads 52 is released, and one of the multiple medicine packages 2 is inserted into the predetermined partition 33 of the multiple medicine dispensing trays 30.

After the above-described operation is performed a plurality of times and a desired one of the multiple medicine packages 2 is inserted into one of the multiple partitions 33 that is at a predetermined position of one of the multiple medicine dispensing trays 30, as illustrated in FIG. 13C, one of the multiple medicine dispensing trays 30 is ejected to the outside of the medication support device 200 through, for example, the second gate 42, and is received by a staff or the like in a day-care center or nursing home or a medication assistant.

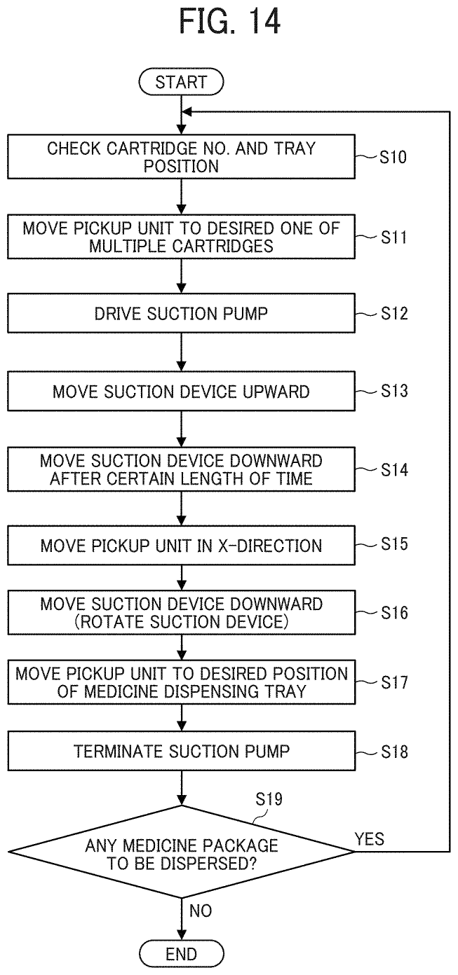

With reference to FIG. 14, supplemental description is given for the operation flow of the pickup unit 50 in an overall operation flow of FIG. 13A-1, FIG. 13A-2, FIG. 13A-3, FIG. 13A-4, FIG. 13A-5, FIG. 13A-6, FIG. 13B and FIG. 13C.

FIG. 14 is a flowchart of the operation flow of the pickup unit 50 according to the present embodiment.

Firstly, in a step S10 of FIG. 14, the number (No.) given to desired one of the multiple cartridges 10 that stores the medicine package 2 to be dispersed is checked, and a desired position of the medicine dispensing tray 30 to which the medicine package 2 is passed is checked.

Subsequently, in a step S11, the pickup unit 50 is moved to desired one of the multiple cartridges 10 by the conveyance operation of the conveyance unit 90. Then, in a step S12, the suction device lifting and lowering motor 63 is driven to rotate while the suction pump 48 is being driven. By so doing, in a step S13, the suction device 51 is moved upward. After a certain length of time has passed after the lowermost one of the multiple medicine packages 2 in one of the multiple cartridges 10 is sucked and held by the pair of suction pads 52, in a step S14, the suction device lifting and lowering motor 63 is driven to rotate in the reverse direction. By so doing, the suction device 51 is moved downward. Subsequently, in a step S15, the pickup unit 50 is moved in the X-direction, and desired one of the multiple medicine packages 2 is completely taken out from one of the multiple cartridges 10. Then, in a step S16, the suction device lifting and lowering motor 63 is further driven to rotate in the reverse direction so as to move the suction device 51 downward. As a result, the suction device 51 is rotated by approximately 90 degrees, and the posture of the medicine package 2 is changed from an approximately parallel or horizontal posture to an approximately vertical posture. Subsequently, the pickup unit 50 is moved to a desired position of one of the multiple medicine dispensing trays 30 by the convey operation of the conveyance unit 90. When the pickup unit 50 has moved to the position of desired one of the multiple medicine dispensing trays 30, in a step S17 and a step S18, the operation of the suction pump 48 is terminated, and the medicine package 2 is released and separated from the pair of suction pads 52. Then, in a step S19, whether there is any medicine package 2 to be dispersed is checked. When there is no medicine package 2 to be dispersed, a series of operation flow is terminated.

On the other hand, when it is determined in the step S19 that there is at least one medicine package 2 to be dispersed, the process is returned to the step S10, and the same series of processes in the above steps are repeated.

In the above embodiments of the present disclosure, the timing at which the posture of one of the multiple medicine packages 2 is changed from a substantially horizontal posture to a substantially vertical posture is immediately after one of the multiple medicine packages 2 to be picked up is completely taken outside the multiple cartridge 10.

With such a configuration and operation, a compact medication support device 200 with reduced width in the side to side direction, which can handle a situation in which there is no room in the lateral direction of the pickup device, can be provided.

When desired one of the multiple medicine packages 2 is picked up from one of the multiple cartridges 10, the pickup unit 50 is positioned under one of the multiple cartridges 10, and such desired one of the multiple medicine packages 2 is taken out in the downward direction of one of the multiple cartridges 10. When one of the multiple medicine packages 2 is taken out from the lower side of one of the multiple cartridges 10 as described above, the next one of the multiple medicine packages 2 automatically moves downward or toward the pack pickup slot 17 due to the self weight of the movable board 16 and the multiple medicine packages 2 left in the cartridge 10. Due to such a configuration, the pickup unit 50 can perform the same operation with a relatively simple configuration regardless of the number of the multiple medicine packages 2 left in the cartridge 10.

According to the above embodiments of the present disclosure, a medication support device can be provided with a relatively simple configuration in which the multiple medicine packs can securely be stored in the multiple containers and those medicine packs can smoothly be picked up on a one-by-one basis with stability when the multiple medicine packs are to be taken out from one of the containers.

More specifically, a medication support device can be provided in which the multiple medicine packs can be held in the container and those medicine packs can easily be taken out and do not fall off easily. Moreover, a medication support device can be provided in which the next one of the multiple medicine packs to be picked up by the pickup device can be prevented from falling off and the posture of the multiple medicine packs do not turn bad when those medicine packs are supported.

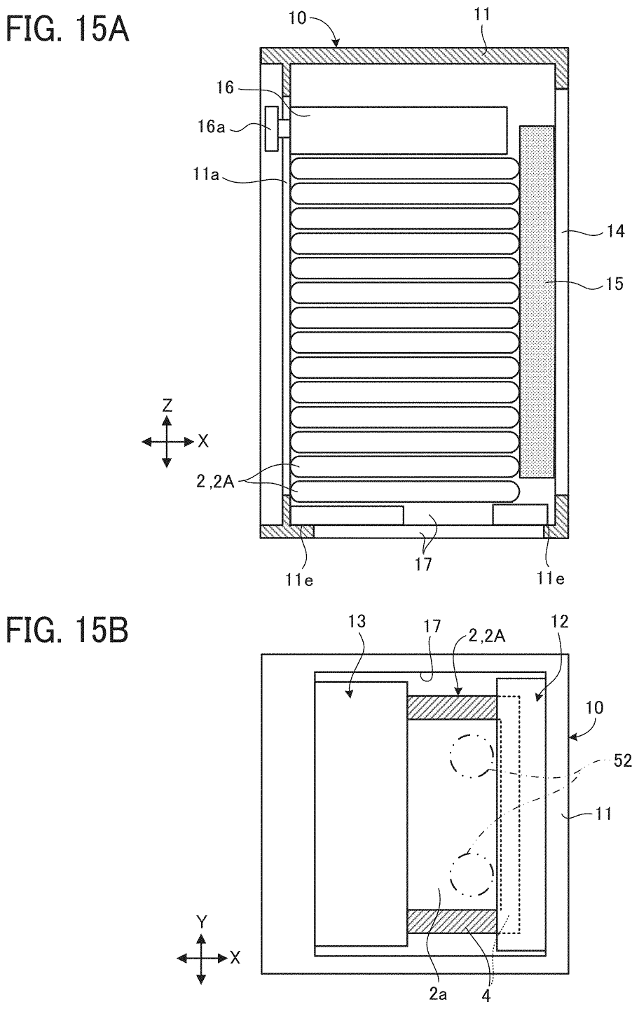

A cartridge 10 according to a modification of the above embodiments of the present disclosure is described below with reference to FIG. 15A and FIG. 15B.

FIG. 15A is a vertical sectional view of the cartridge 10 according to a modification of the above embodiments of the present disclosure.

FIG. 15B is a bottom view of the cartridge 10 of FIG. 15A according to the present modification of the above embodiments of the present disclosure.

In the multiple cartridges 10 illustrated in FIG. 4A, FIG. 4B, and FIG. 5, embodiments in which the right support portion 12, the left support portion 13, and the casing 11 are formed separately from each other are described. However, no limitation is indicated thereby, and the right support portion 12, the left support portion 13, and the casing 11 may integrally be formed as illustrated in FIG. 15A and FIG. 15B. For example, the casing 11 may be formed by a machine that adopts injection molding, using a desired kind of resin. By so doing, the casing 11 can be integrally molded together with the right support portion 12 and the left support portion 13 as illustrated in FIG. 4A and FIG. 4B.

According to the present modification of the above embodiments of the present disclosure described with reference to FIG. 15A and FIG. 15B, the cartridge can be configured or formed more easily.

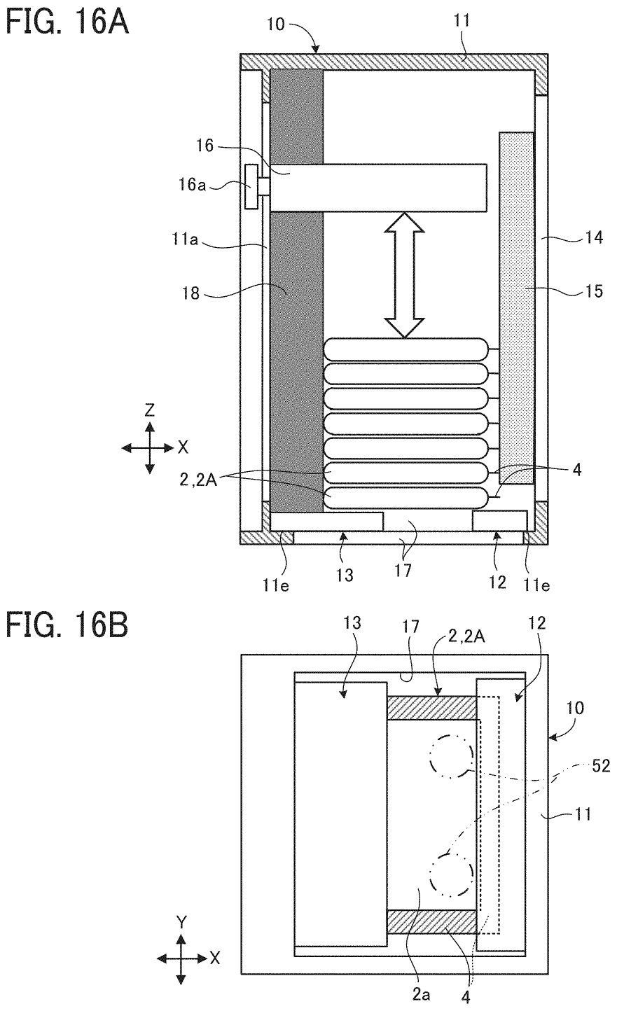

FIG. 16A is a vertical sectional view of the cartridge 10 according to another modification of the above embodiments of the present disclosure different from the modifications illustrated in FIG. 15A and FIG. 15B.

FIG. 16B is a bottom view of the cartridge 10 of FIG. 16A, according to the present modification of the above embodiments of the present disclosure.

In FIG. 16B, the pressure-bonded portions 4 are also illustrated in order to clarify the pack size of the multiple medicine packages 2 and the bound packages 2A. In the following description, the case of the multiple medicine packages 2 is described.

When the sizes of the multiple medicine packages 2, which correspond to the lengths of packs in the X-direction in FIG. 16A and FIG. 16B, are different from the sizes of the multiple medicine package 2 illustrated in FIG. 4A and FIG. 4B, the positions of the multiple medicine packages 2 that are stored in one of the multiple cartridges 10 are not stabilized, and the positions at which one of the multiple medicine packages 2 is sucked by the pair of suction pads 52 (see FIG. 8A and FIG. 8B) tend to vary.

In order to deal with such a situation, as illustrated in FIG. 16A, each one of the multiple cartridges 10 is provided with a pack-length adjuster 18 on the other side of sides of the multiple medicine packages 2 to be sucked by the pair of suction pads 52. Accordingly, the positions at which each one of the packs are sucked by the pair of suction pads 52 can be made constant regardless of the sizes of the multiple medicine packages 2. The pack-length adjuster 18 is arranged on the inner wall of the casing 11 in a detachable manner and may be replaced only when the size of the packs in use is to be changed.

According to the present modification of the above embodiments of the present disclosure described with reference to FIG. 16A and FIG. 16B, the position of the multiple packs that are held in the cartridge can consistently be aligned at the position where each one of the multiple packs is to be sucked by the suction device.

The above embodiments of the present disclosure described above substantially include, for example, the following modifications.

In particular, a medication support device according to a first mode of the present disclosure includes a container such as the cartridge 10 that stores a plurality of medicine packs stacked on top of each other in layers, a pickup device such as the pickup unit 50 configured to pick up a specific one of the multiple medicine packs from the container, a conveyor such as the conveyance unit 90 that conveys the specific one of the plurality of medicine packs picked up by the pickup device, a medicine dispenser such as the medicine dispensing tray in which the specific one of the plurality of medicine packs conveyed by the conveyor are arranged, and a pack passing-through portion such as the pack pickup slot 17 disposed on a bottom of the container, where the specific one of the plurality of medicine packs picked up by the pickup device passes through the pack passing-through portion. In the medication support device according to the first mode of the present disclosure, the pack passing-through portion has a plurality of support portions such as the right support portion 12 and the left support portion 13 that support the medicine pack. In the medication support device according to the first mode of the present disclosure, the multiple support portions are fixation members fixed to the pack passing-through portion, and the multiple support portions vary in shape.

With the medication support device according to the first mode of the present disclosure, a medication support device can be provided with a relatively simple configuration in which the multiple medicine packs can securely be stored in the multiple containers and those medicine packs can smoothly be picked up on a one-by-one basis with stability when the multiple medicine packs are to be taken out from one of the containers.

More specifically, a medication support device can be provided in which the multiple medicine packs can be held in the container and those medicine packs can easily be taken out and do not fall off easily. Moreover, a medication support device can be provided in which the next one of the multiple medicine packs to be picked up by the pickup device can be prevented from falling off and the posture of the multiple medicine packs do not turn bad when those medicine packs are supported.

According to the second mode of the present disclosure, in the medication support device according to the first mode of the present disclosure, the multiple support portions (12, 13) have different heights to support the multiple medicine packs (2).

With the medication support device according to the second mode of the present disclosure, the multiple medicine packs each of which has uneven thickness can be stably supported.

According to the third mode of the present disclosure, in the medication support device according to the first or second mode of the present disclosure, the pickup device includes a suction device such as the suction device 51 according to the above embodiments of the present disclosure configured to suck and pick up one of the plurality of medicine packs, and one of the multiple support portions proximate to a portion of one of the multiple medicine packs to be sucked by the suction device has a greater height than another one of the multiple support portions.

With the medication support device according to the third mode of the present disclosure, for example, by increasing the height of the multiple medicine packs at the portions having a small thickness as the packs are stapled as bound packages 2A on that side, the posture of the multiple medicine packs in the container can be stabilized when those medicine packs are supported. Accordingly, an error in sucking one of the medicine packs can be prevented.

According to the fourth mode of the present disclosure, in the medication support device according to any one of the first mode to the third mode of the present disclosure, the multiple support portions are disposed separately from each other.

According to the fifth mode of the present disclosure, in the medication support device according to any one of the first mode to the third mode of the present disclosure, the multiple support portions are formed as a single integrated unit. With the medication support device according to the fifth mode of the present disclosure, the multiple support portions can be configured or formed easily.

According to the sixth mode of the present disclosure, in the medication support device according to any one of the first mode to the fifth mode of the present disclosure, the multiple support portions (12, 13) have different lengths to support the multiple medicine packs (2). With the medication support device according to the sixth mode of the present disclosure, one of the multiple medicine packs can smoothly be taken out.

According to the seventh mode of the present disclosure, in the medication support device according to any one of the first mode to the sixth mode of the present disclosure, the pickup device includes a suction device that sucks and picks up one of the plurality of medicine packs. In the medication support device according to the seventh mode of the present disclosure, one of the multiple support portions proximate to a portion of one of the multiple medicine packs to be sucked by the suction device has a smaller length than a length of another one of the support portions proximate to a portion of the one of the multiple medicine packs not to be sucked by the suction device.

With the medication support device according to the seventh mode of the present disclosure, one of the multiple medicine packs can smoothly be sucked up and pulled out.

According to the eighth mode of the present disclosure, in the medication support device according to any one of the first mode to the seventh mode of the present disclosure, the pickup device includes a suction device that sucks up and picks up one of the multiple medicine packs. In the medication support device according to the eighth mode of the present disclosure, the container has a pack-length adjuster disposed adjacent to a portion of the multiple medicine packs on a reverse side of another portion of the multiple medicine packs to be sucked by the suction device, and the pack-length adjuster is configured to control a length of the multiple medicine packs.

With the medication support device according to the eighth mode of the present disclosure, the position of the multiple medicine packs that are held in the container can consistently be aligned adjacent to an end face where each one of the multiple packs is to be sucked by the suction device.

According to the ninth mode of the present disclosure, in the medication support device according to any one of the first mode to the eighth mode of the present disclosure, the pickup device includes a suction device that sucks and picks up one of the plurality of medicine packs. In the medication support device according to the ninth mode of the present disclosure, the container has a pressing member movable downward in a vertical direction, and the pressing member is configured to press down the multiple medicine packs stored in the container by its own weight or self weight.

With the medication support device according to the ninth mode of the present disclosure, the multiple medicine packs that are stored in the container smoothly move downward to the lowermost portion of the container where one of the multiple medicine packs is to be sucked up and pulled out, and the posture of the multiple medicine packs in the container can be stabilized when those medicine packs are supported. Accordingly, an error in sucking one of the medicine packs can be prevented. According to the tenth mode of the present disclosure, in the medication support device according to any one of the first mode to the ninth mode of the present disclosure, the multiple medicine packs include a plurality of medicine packages in each of which a plurality of medicines are packed and bound packages in which the multiple medicine packages are stacked on top of each other in layers and bound together.

With the medication support device according to the tenth mode of the present disclosure, various types of medicines in various kinds of states can be provided based on the demands from patients who take medicines or medication assistants.

Note that numerous additional modifications and variations are possible in light of the above teachings. It is therefore to be understood that within the scope of the appended claims, the embodiments of the present disclosure may be practiced otherwise than as specifically described herein. For example, elements and/or features of different illustrative embodiments may be combined with each other and/or substituted for each other within the scope of this disclosure and appended claims.

Any one of the above-described operations may be performed in various other ways, for example, in an order different from the one described above.

Each of the functions of the described embodiments may be implemented by one or more processing circuits or circuitry. Processing circuitry includes a programmed processor, as a processor includes circuitry. A processing circuit also includes devices such as an application-specific integrated circuit (ASIC), digital signal processor (DSP), field-programmable gate array (FPGA), and conventional circuit components arranged to perform the recited functions.

What is claimed is:

1. A medication support device comprising:
a container that stores a plurality of medicine packs stacked on top of each other in layers;
a pickup device configured to pick up a specific one of the plurality of medicine packs from the container;
a conveyor configured to convey the specific one of the plurality of medicine packs picked up by the pickup device;
a medicine dispenser configured to arrange the specific one of the plurality of medicine packs conveyed by the conveyor; and
a pack passing-through portion disposed on a bottom of the container, the specific one of the plurality of medicine packs picked up by the pickup device passing through the pack passing-through portion, the pack passing-through portion having a plurality of support portions that support the plurality of medicine packs, the plurality of support portions being fixation members fixed to the pack passing-through portion, the plurality of support portions varying in shape.

2. The medication support device according to claim 1,
wherein the plurality of support portions (12, 13) have different heights to support the plurality of medicine packs (2).

3. The medication support device according to claim 1,
wherein the pickup device includes a suction device configured to suck and pick up one of the plurality of medicine packs, and
wherein one of the plurality of support portions proximate to a portion of one of the plurality of medicine packs to be sucked by the suction device has a greater height than another one of the plurality of support portions.

4. The medication support device according to claim 1,
wherein the plurality of support portions are disposed separately from each other.

5. The medication support device according to claim 1,
wherein the plurality of support portions are a single integrated unit.

6. The medication support device according to claim 1,
wherein the plurality of support portions have different lengths to support the plurality of medicine packs.

7. The medication support device according to claim 1,
wherein the pickup device includes a suction device configured to suck and pick up one of the plurality of medicine packs, and
wherein one of the plurality of support portions proximate to a portion of one of the plurality of medicine packs to be sucked by the suction device has a smaller length than a length of another one of the support portions proximate to a portion of the one of the plurality of medicine packs not to be sucked by the suction device.

8. The medication support device according to claim 1,
wherein the pickup device includes a suction device configured to suck and pick up one of the plurality of medicine packs,
wherein the container has a pack-length adjuster disposed adjacent to a portion of the plurality of medicine packs on a reverse side of another portion of the plurality of medicine packs to be sucked by the suction device, and the pack-length adjuster is configured to control a length of the plurality of medicine packs.

9. The medication support device according to claim 1,
wherein the pickup device includes a suction device configured to suck and pick up one of the plurality of medicine packs,
wherein the container has a pressing member movable downward in a vertical direction, and the pressing member is configured to press down the plurality of medicine packs stored in the container by self weight.

10. The medication support device according to claim 1,
wherein the plurality of medicine packs include a plurality of medicine packages in each of which a plurality of medicines are packed and bound packages in which the plurality of medicine packages are stacked on top of each other in layers and bound together.
